(19) United States
(12) Patent Application Publication
(10) Pub. No.: US 2025/0108213 A1
(43) Pub. Date: Apr. 3, 2025

(54) COMPOSITIONS AND METHODS FOR ENHANCING THE EFFECT OF PLATINUM-BASED SYSTEMIC THERAPY

(71) Applicant: NOVOCURE GMBH, Root D4 (CH)

(72) Inventor: Eyal Dor-On, Haifa (IL)

(21) Appl. No.: 18/899,404

(22) Filed: Sep. 27, 2024

Related U.S. Application Data
(60) Provisional application No. 63/586,738, filed on Sep. 29, 2023.

(51) Int. Cl. A61N 1/36; A61K 31/282; A61K 31/4439; A61K 31/501; A61K 31/502; A61K 31/55; A61K 31/555; A61P 35/00

(52) U.S. Cl. CPC A61N 1/36002; A61K 31/282; A61K 31/4439; A61K 31/501; A61K 31/502; A61K 31/55; A61K 31/555; A61P 35/00

(57) ABSTRACT

Disclosed are methods of enhancing the effect of a platinum-based systemic therapy in a subject having cancer comprising applying an alternating electric field to a target site of the subject for a period of time, the alternating electric field having a frequency and field strength, wherein the target site comprises one or more cancer cells, and administering a therapeutically effective amount of a platinum-based systemic therapy to the subject, wherein the cancer is resistant to platinum-based systemic therapy. Disclosed are methods of enhancing the effect of a PARP inhibitor treatment in a subject having cancer comprising applying an alternating electric field to a target site of the subject for a period of time, the alternating electric field having a frequency and field strength, wherein the target site comprises one or more cancer cells, and administering a therapeutically effective amount of a PARP inhibitor to the subject, wherein the cancer is resistant to platinum-based systemic therapy, resistant to a PARP inhibitor, or both. Disclosed are also methods of treating a subject and increasing apoptosis using an alternating electric field in combination with a platinum-based system therapy and/or a PARP inhibitor.

### CROSS-REFERENCE TO RELATED APPLICATIONS

This application is claims the benefit of U.S. Provisional Patent Application No. 63/586,738, filed Sep. 29, 2023, each of which is incorporated by reference herein in its entirety.

### BACKGROUND

Platinum-based chemotherapy is recommended after surgery for most patients with ovarian cancer; and PARP inhibitors (PARPi) are recommended as maintenance therapy. In some aspects, a cancer can become resistant to one or both of platinum-based chemotherapy and PARP inhibitors.

Tumor Treating Fields (TTFields) or alternating electric fields are electric fields that exert physical forces to disrupt cellular processes critical for cancer cell viability and tumor progression and have been shown to induce a state of BRCAness, a defect in homologous recombination repair, in various cancer type.

### BRIEF SUMMARY

Disclosed are methods of enhancing the effect of a platinum-based systemic therapy in a subject having cancer comprising applying an alternating electric field to a target site of the subject for a period of time, the alternating electric field having a frequency and field strength, wherein the target site comprises one or more cancer cells, and administering a therapeutically effective amount of a platinum-based systemic therapy to the subject, wherein the one or more cancer cells are resistant to a platinum-based systemic therapy.

Disclosed are methods of enhancing the effect of a PARP inhibitor treatment in a subject having cancer comprising applying an alternating electric field to a target site of the subject for a period of time, the alternating electric field having a frequency and field strength, wherein the target site comprises one or more cancer cells, and administering a therapeutically effective amount of a PARP inhibitor to the subject, wherein the one or more cancer cells are resistant to a platinum-based systemic therapy, resistant to a PARP inhibitor, or both.

Disclosed are methods of treating a subject having cancer comprising applying an alternating electric field to a target site of the subject for a period of time, the alternating electric field having a frequency and field strength, wherein the target site comprises one or more cancer cells, and administering a therapeutically effective amount of a platinum-based systemic therapy to the subject, wherein the one or more cancer cells are resistant to platinum-based systemic therapy.

Disclosed are methods of treating a subject having cancer comprising applying an alternating electric field to a target site of the subject for a period of time, the alternating electric field having a frequency and field strength, wherein the target site comprises one or more cancer cells, and administering a therapeutically effective amount of a PARP inhibitor to the subject. In some aspects, the one or more cancer cells are resistant to platinum-based systemic therapy. In some aspects, the one or more cancer cells are resistant to a PARP inhibitor. In some aspects, one or more cancer cells are resistant to a platinum-based systemic therapy and a PARP inhibitor.

Disclosed are methods of increasing apoptosis in a cell comprising applying an alternating electric field to a cancer cell for a period of time, the alternating electric field having a frequency and field strength, and administering a therapeutically effective amount of a platinum-based systemic therapy to the cell, wherein the cell is resistant to a platinum-based systemic therapy.

Disclosed are methods of increasing apoptosis in a cell comprising applying an alternating electric field to a cell for a period of time, the alternating electric field having a frequency and field strength, and administering a therapeutically effective amount of a PARP inhibitor to the cell. In some aspects, the cell is resistant to a platinum-based systemic therapy. In some aspects, the cell is resistant to a PARP inhibitor.

Additional advantages of the disclosed methods and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed methods and compositions and together with the description, serve to explain the principles of the disclosed methods and compositions.

FIG. 1 shows TTFields augmented the cytotoxic effect (examined by flow cytometer cell count) of olaparib and niraparib, synergistically, in A2780 cells (possessing BRCA wt) and additively in OVCAR-3 (possessing BRCA mutant) cells; Signs of synergy were seen for TTFields with carboplatin in A2780.

FIG. 2 shows TTFields synergistically elevated the overall effects (examined by calculation of cell count and colonogenic effect) of olaparib and niraparib in A2780 cells and in an additive manner in OVCAR-3 cells; Signs of synergy were seen for TTFields with carboplatin in A2780.

FIG. 3 shows TTFields enhanced the apoptotic effect (examined by 7AAD/Annexin V staining and read in flow cytometer) of olaparib, niraparib, and carboplatin in A2780 and OVCAR-3 cells.

FIG. 4 shows a treatment paradigm for epithelial ovarian cancer.

FIG. 5 shows mechanisms of platinum resistance.

FIG. 6 shows overlapping mechanisms of resistance to platinum and PARPi treatment.

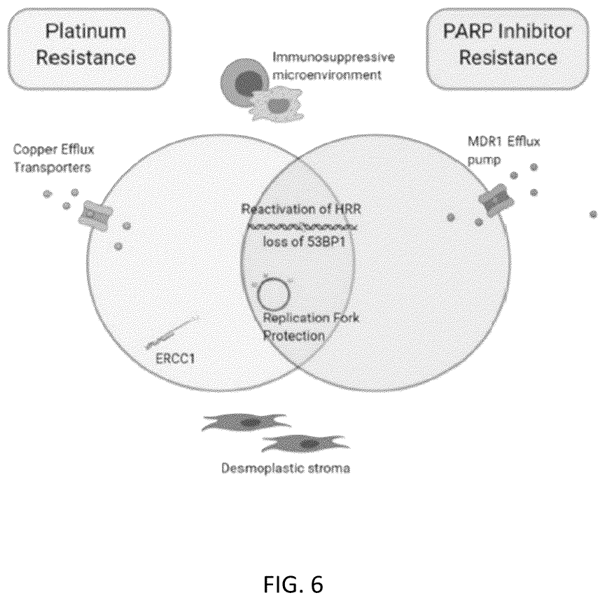

FIG. 7 shows objectives to study the effect of TTFields on cytotoxicity, apoptosis and clonogenic survival in a carboplatin-resistant ovarian cell line.

FIG. 8 shows A2780cis cells show a 5-fold decreased sensitivity to carboplatin compared with wild type A2780 cells.

FIG. 9 shows TTFields synergistically increase the effect of carboplatin in A2780cis cells compared to an additive effect in wild type A2780 cells.

FIG. 10 shows A2780cis cell line shows a ±9-fold reduced sensitivity to PARPi in A2780cis cells compared with A2780 wild type cells.

FIG. 11 shows TTFields enhances the effect of PARPi in A2780cis cells compared with A2780 wild type cells.

FIG. 12 shows cell count of A2780, OVCAR-3, and A2780cis human ovarian cancer cells (HRP, HRD, and platinum-resistant cells, respectively) following 72 h treatment with various carboplatin doses.

FIGS. 13A-J show TTFields enhance the efficacy of carboplatin, additively in HRD cells and with a tendency to synergy in HRP and platinum-resistant cells.

FIGS. 14A-B show Olaparib and niraparib dose response curves in the three different cell lines.

FIGS. 15A-J show TTFields enhance the efficacy of olaparib, additively in HRD cells, with a tendency to synergy in platinum-resistant cells, and with high synergy in HRP cells.

FIGS. 16A-J show TTFields enhance the efficacy of niraparib, additively in HRD cells, with a tendency to synergy in platinum-resistant cells, and with high synergy in HRP cells.

FIGS. 17A-N show TTFields increase DNA damage induced by carboplatin and PARPi, elevate levels of p21, and downregulate the FA-BRCA pathway.

FIGS. 18A-I show TTFields support drug-felicitated G2/M cell cycle arrest in response to the induced DNA damage.

FIGS. 19A-R show complementary data for the cell cycle analysis. Percentage of cells in G0/G1 and S phases for FIG. 18A-I.

FIGS. 20A-D show TTFields co-treatment with olaparib inhibits tumor growth and prolongs survival in ovarian cancer bearing mice.

FIG. 21 shows complementary data for in vivo study. Total photon flux of the bioluminescent imaging analysis before and after treatment.

### DETAILED DESCRIPTION

The disclosed methods and compositions may be understood more readily by reference to the following detailed description of particular embodiments and the Example included therein and to the Figures and their previous and following description.

It is to be understood that the disclosed methods and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a peptide is disclosed and discussed and a number of modifications that can be made to a number of molecules including the amino acids are discussed, each and every combination and permutation of the peptide and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed, it is understood that each of these additional steps can be performed with any specific embodiment or combination of embodiments of the disclosed methods, and that each such combination is specifically contemplated and should be considered disclosed.

### A. Definitions

It is understood that the disclosed methods and compositions are not limited to the particular methodology, protocols, and reagents described as these may vary. It is also to be understood that the terminology used herein is for the purpose of describing particular embodiments only, and is not intended to limit the scope of the present invention, which will be limited only by the appended claims.

It must be noted that as used herein and in the appended claims, the singular forms “a”, “an”, and “the” include plural reference unless the context clearly dictates otherwise. Thus, for example, reference to “a platinum-based systemic therapy” includes a plurality of such therapies, reference to “the subject” is a reference to one or more subjects and equivalents thereof known to those skilled in the art, and so forth.

The word “or” as used herein means any one member of a particular list and also includes any combination of members of that list.

As used herein, a “target site” is a specific site or location within or present on a subject or patient. For example, a “target site” can refer to, but is not limited to a cell (e.g., a cancer cell), population of cells, organ, tissue, or a tumor. Thus, the phrase “target cell” can be used to refer to target site, wherein the target site is a cell. In some aspects, a “target cell” can be a cancer cell. In some aspects, organs that can be target sites include, but are not limited to, the brain. In some aspects, a cell or population of cells that can be a target site or a target cell include, but are not limited to, a cancer cell (e.g., an ovarian cancer cell). In some aspects, a “target site” can be a tumor target site.

A “tumor target site” is a site or location within or present on a subject or patient that comprises or is adjacent to one or more cancer cells, previously comprised one or more tumor cells, or is suspected of comprising one or more tumor cells. For example, a tumor target site can refer to a site or location within or present on a subject or patient that is prone to metastases. Additionally, a target site or tumor target site can refer to a site or location of a resection of a primary tumor within or present on a subject or patient. Additionally, a target site or tumor target site can refer to a site or location adjacent to a resection of a primary tumor within or present on a subject or patient.

As used herein, an “alternating electric field” or “alternating electric fields” refers to a very-low-intensity, directional, intermediate-frequency alternating electric fields delivered to a subject, a sample obtained from a subject or to a specific location within a subject or patient (e.g., a target site such as a cell). In some aspects, the alternating electric field can be in a single direction or multiple directions. In some aspects, alternating electric fields can be delivered through two pairs of transducer arrays that generate perpendicular fields within the target site. For example, for the Optune™ system (an alternating electric fields delivery system) one pair of electrodes is located to the left and right (LR) of the target site, and the other pair of electrodes is located anterior and posterior (AP) to the target site. Cycling the field between these two directions (i.e., LR and AP) ensures that a maximal range of cell orientations is targeted.

As used herein, an “alternating electric field” applied to a tumor target site can be referred to as a “tumor treating field” or “TTField.” TTFields have been established as an anti-mitotic cancer treatment modality because they interfere with proper micro-tubule assembly during metaphase and eventually destroy the cells during telophase, cytokinesis, or subsequent interphase. TTFields target solid tumors and are described in U.S. Pat. No. 7,565,205, which is incorporated herein by reference in its entirety for its teaching of TTFields

In-vivo and in-vitro studies show that the efficacy of TTFields therapy increases as the intensity of the alternating electric field increases. Therefore, optimizing array placement on a subject to increase the intensity in the target site or target cell is standard practice for the Optune system. Array placement optimization may be performed by “rule of thumb” (e.g., placing the arrays on the subject as close to the target site or target cell as possible), measurements describing the geometry of the patient's body, target site dimensions, and/or target site or cell location. Measurements used as input may be derived from imaging data. Imaging data is intended to include any type of visual data, such as for example, single-photon emission computed tomography (SPECT) image data, x-ray computed tomography (x-ray CT) data, magnetic resonance imaging (MRI) data, positron emission tomography (PET) data, data that can be captured by an optical instrument (e.g., a photographic camera, a charge-coupled device (CCD) camera, an infrared camera, etc.), and the like. In certain implementations, image data may include 3D data obtained from or generated by a 3D scanner (e.g., point cloud data). Optimization can rely on an understanding of how the alternating electric field distributes within the target site or target cell as a function of the positions of the array and, in some aspects, take account for variations in the electrical property distributions within the heads of different patients.

The term “subject” refers to the target of administration, e.g., an animal. Thus, the subject of the disclosed methods can be a vertebrate, such as a mammal. For example, the subject can be a human. The term does not denote a particular age or sex. Subject can be used interchangeably with “individual” or “patient.” For example, the subject of administration can mean the recipient of the alternating electrical field. For example, the subject of administration can be a subject with ovarian cancer or lung cancer.

By “treat” is meant to administer or apply a therapeutic, such as an alternating electric field and a platinum-based systemic therapy or a PARP inhibitor, to a subject, such as a human or other mammal (for example, an animal model), that has cancer or has an increased susceptibility for developing cancer, in order to prevent or delay a worsening of the effects of the disease or infection, or to partially or fully reverse the effects of cancer. For example, treating a subject having glioblastoma can comprise delivering a therapeutic to a cell in the subject.

As used herein, the term “cancer” refers to one or more cancer cells.

By “prevent” is meant to minimize or decrease the chance that a subject develops cancer.

As used herein, the terms “administering” and “administration” refer to any method of providing a therapy (e.g., a platinum-based systemic therapy or a PARP inhibitor) to a subject directly or indirectly to a target site. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a therapy (e.g., a platinum-based systemic therapy or a PARP inhibitor) can be administered therapeutically; that is, administered to treat cancer. In further various aspects, a therapy (e.g., a platinum-based systemic therapy or a PARP inhibitor) can be administered prophylactically; that is, administered for prevention of cancer. In an aspect, the skilled person can determine an efficacious dose, an efficacious schedule, or an efficacious route of administration so as to treat a subject. In some aspects, administering comprises contacting, exposing or applying. Thus, in some aspects, exposing a target site or subject to an alternating electric field or applying alternating electrical fields to a target site or subject or contacting an alternating electric field to a target site or subject means administering an alternating electric field to the target site or subject. In some aspects, contacting, exposing and applying can be used interchangeably.

As used herein, the term “therapeutically effective amount” means an amount of a therapeutic (e.g., platinum-based systemic therapy, PARP inhibitor) that is sufficient, when administered to a subject suffering from or susceptible to a disease, disorder, and/or condition, to treat, alleviate, ameliorate, relieve, alleviate symptoms of, prevent, delay onset of, inhibit progression of, reduce severity of, and/or reduce incidence of the disease, disorder, and/or condition. In some aspects, a therapeutically effective amount of a therapeutic (e.g. platinum-based systemic therapy, PARP inhibitor) is an amount that, when administered together with application of an alternating electric field, would not have a therapeutic benefit in a subject when the same amount of therapeutic is administered without an alternating electric field. For example, in some aspects, a therapeutically effective amount of a platinum based systemic therapy is not effective in a subject having a cancer resistant to the platinum based systemic therapy.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed methods and compositions belong. Although any methods and materials similar or equivalent to those described herein can be used in the practice or testing of the present methods and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Alternating Electric Fields

The methods disclosed herein comprise applying an alternating electric field. In some aspects, the alternating electric field used in the methods disclosed herein is a tumor-treating field. In some aspects, the alternating electric field can vary dependent on the type of cell or condition to which the alternating electric field is applied. In some aspects, the alternating electric field can be applied through one or more electrodes placed on or in the subject's body. In some aspects, there can be two or more pairs of electrodes. For example, arrays can be placed on the front/back and sides of a patient and can be used with the systems and methods disclosed herein. In some aspects, where two pairs of electrodes are used, the alternating electric field can alternate between the pairs of electrodes. For example, a first pair of electrodes can be placed on the front and back of the subject and a second pair of electrodes can be placed on either side of the subject, the alternating electric field can then be applied and can alternate between the front and back electrodes and then to the side to side electrodes.

In some aspects, the frequency of the alternating electric field is between 100 and 500 kHz. In some aspects, the frequency of the alternating electric field is between 50 kHz and 1 MHz. The frequency of the alternating electric fields can also be, but is not limited to, between 50 and 500 kHz, between 100 and 500 kHz, between 25 kHz and 1 MHz, between 50 and 190 kHz, between 25 and 190 kHz, between 180 and 220 kHz, or between 210 and 400 kHz. In some aspects, the frequency of the alternating electric field can be about 50 kHz, 100 kHz, 150 kHz, 200 kHz, 250 kHz, 300 kHz, 350 kHz, 400 kHz, 450 kHz, 500 kHz, or any frequency between. In some aspects, the frequency of the alternating electric field is from about 200 kHz to about 400 kHz, from about 250 kHz to about 350 kHz, and may be about 150 kHz, 200 kHz, or 300 kHz.

In some aspects, the field strength of the alternating electric fields can be between 0.5 and 4 V/cm RMS. In some aspects, the field strength of the alternating electric field can be between 1 and 4 V/cm RMS. In some aspects, different field strengths can be used (e.g., between 0.1 and 10 V/cm). In some aspects, the field strength can be about 1.75 V/cm RMS. In some embodiments the field strength is at least 1 V/cm RMS. In some aspects, the field strength can be about 0.9 V/cm RMS. In other embodiments, combinations of field strengths are applied, for example combining two or more frequencies at the same time, and/or applying two or more frequencies at different times.

In some aspects, the alternating electric field can be applied for a variety of different intervals ranging from 0.5 hours to 72 hours. In some aspects, a different duration can be used (e.g., between 0.5 hours and 14 days). In some aspects, application of the alternating electric field can be repeated periodically. For example, the alternating electric field can be applied every day for a two hour duration.

In some aspects, the exposure may last for at least 6 hours, at least 12 hours, at least 24 hours, at least 36 hours, at least 48 hours, or at least 72 hours or more.

In addition, when the alternating electric field is applied to a subject, the period of time that the alternating electric field is applied may be a continuous period of time or a cumulative period of time. That is, the period of time that the alternating electric field is applied may include a single session (i.e., continuous application) as well as multiple sessions with minor breaks in between sessions (i.e., consecutive applications for a cumulative period). For example, a subject is allowed to take breaks during treatment with an alternating electric field device and is only expected to have the device positioned on the body and operational for at least about 50%, at least about 60%, at least about 70%, or at least about 80% of the total treatment period (e.g., over a course of one day, one week, two weeks, one month, two months, three months, four months, five months, etc.). For example, the alternating electric field can be applied for at least 12 hours, 16 hours, or 18 hours cumulative each day for a week, a month, two months, three months, etc.

The disclosed methods comprise applying one or more alternating electric fields to a cell or to a subject. In some aspects, the alternating electric field is applied to a target site or tumor target site. When applying an alternating electric field to a cell, this can often refer to applying an alternating electric field to a subject comprising a cell. Thus, applying an alternating electric field to a target site of a subject results in applying an alternating electric field to a cell.

### C. Pharmaceutical Compositions

Disclosed are pharmaceutical compositions and formulations comprising one or more platinum-based systemic therapies. In some aspects, the platinum-based systemic therapy can be, but is not limited to, nedaplatin, satraplatin, cisplatin, carboplatin, oxaliplatin, phenanthriplatin, picoplatin, or triplatin tetranitrate. In some aspects, the compositions and formulations comprise one or more PARP inhibitors. In some aspects, the compositions and formulations comprise one or more platinum-based systemic therapy and PARP inhibitor.

In some instances, the compositions can further comprise a pharmaceutically acceptable carrier. By “pharmaceutically acceptable” is meant a material or carrier that would be selected to minimize any degradation of the active ingredient and to minimize any adverse side effects in the subject, as would be well known to one of skill in the art. As used herein, the term “pharmaceutically acceptable carrier” refers to sterile aqueous or nonaqueous solutions, dispersions, suspensions or emulsions, nanoparticles, liposomes, gels as well as sterile powders for reconstitution into sterile injectable solutions or dispersions just prior to use. Examples of suitable pharmaceutically acceptable carriers are known to persons of skill in the art.

Pharmaceutical compositions can also include carriers, thickeners, diluents, buffers, preservatives and the like, as long as the intended activity of the platinum-based systemic therapy or PARP inhibitor is not compromised. Pharmaceutical compositions may also include one or more active ingredients (in addition to the platinum-based systemic therapy or PARP inhibitor) such as antimicrobial agents, anti-inflammatory agents, anesthetics, and the like. The pharmaceutical composition may be administered in a number of ways depending on whether local or systemic treatment is desired, and on the area to be treated.

In certain aspects, the disclosed pharmaceutical compositions comprise the disclosed platinum-based systemic therapy as an active ingredient, a pharmaceutically acceptable carrier, and, optionally, other therapeutic ingredients or adjuvants. The instant compositions include those suitable for nasal, oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

### D. Methods

Disclosed are several methods wherein the cell or subject can be any of those described in any of the sections throughout. For example, a subject administered a platinum-based therapy in the methods of enhancing platinum-based therapy can be the same type of subject used in any of the other methods described herein.

In some aspects of the disclosed methods, administering a therapeutically effective amount of a platinum-based systemic therapy and/or a PARP inhibitor to a subject results in administration of the platinum-based systemic therapy and/or PARP inhibitor to the target site (i.e., the one or more cancer cells) where the alternating electric field is applied. Thus, in some aspects, the platinum-based systemic therapy and/or PARP inhibitor are directly administered to the target site or indirectly administered to the target site, both resulting in the platinum-based systemic therapy and/or a PARP inhibitor reaching the target site.

### 1. Methods of Enhancing Platinum-Based Therapy

Disclosed are methods of enhancing the effect of a platinum-based systemic therapy in a subject having cancer comprising applying an alternating electric field to a target site of the subject for a period of time, the alternating electric field having a frequency and field strength, wherein the target site comprises one or more cancer cells, and administering a therapeutically effective amount of a platinum-based systemic therapy to the subject, wherein the one or more cancer cells are resistant to platinum-based systemic therapy. Thus, in some aspects, the use of an alternating electric field can alter the subject's cells enough to convert cancer cells resistant to platinum-based systemic therapy to cancer cells treatable with a platinum-based systemic therapy. In some aspects, a therapeutically effective amount of a platinum-based systemic therapy, when administered together with application of an alternating electric field, would not have a therapeutic benefit in this patient population when the same amount of platinum-based systemic therapy is administered without an alternating electric field.

In some aspects, a cancer (e.g., one or more cancer cells) resistant to platinum-based systemic therapy can be a cancer that has progressed while on or after treatment with a platinum-based systemic therapy. In some aspects, a cancer resistant to platinum-based systemic therapy can be a cancer that never responded to a platinum-based systemic therapy. In some aspects, a cancer resistant to platinum-based systemic therapy can be a cancer that never responded to a platinum-based systemic therapy.

In some aspects, the subject having cancer has a cancer that has progressed on or after treatment with a platinum-based systemic therapy. In some aspects, the cancer has progressed less than one month after stopping platinum-based systemic therapy, between one and six months after stopping platinum-based systemic therapy, between six months and 12 months after stopping platinum-based systemic therapy, or more than 12 months after stopping platinum-based systemic therapy.

In some aspects, the subject has been diagnosed with radiological progression while on or after platinum-based systemic therapy.

In some aspects, the platinum-based systemic therapy can be, but is not limited to, one or more of nedaplatin, satraplatin, cisplatin, carboplatin, oxaliplatin, phenanthriplatin, picoplatin, and triplatin tetranitrate. Thus, in some aspects, a subject resistant to platinum-based systemic therapy is resistant to the one or more of nedaplatin, satraplatin, cisplatin, carboplatin, oxaliplatin, phenanthriplatin, picoplatin, and triplatin tetranitrate.

In some aspects, the subject having cancer has ovarian cancer, breast cancer, pancreatic cancer, cervical cancer, brain cancer, hepatobiliary cancer, prostate cancer, head and neck cancers, glioblastoma, gliosarcoma, leukemia, or non-small cell lung cancer. In some aspects, the cancer can be, but is not limited to, ovarian cancer, hepatobiliary cancer, prostate cancer, pancreatic cancer, cervical cancer, head and neck cancers, glioblastoma, gliosarcoma, leukemia, or non-small cell lung cancer. Thus, in some aspects, the cancer cells are derived from one or more of these cancers.

In some aspects, the target site comprises one or more ovarian cancer cells. In some aspects, the target site comprises one or more breast cancer cells, pancreatic cancer cells, cervical cancer cells, brain cancer cells, hepatobiliary cancer cells, prostate cancer cells, head and neck cancer cells, glioblastoma cells, gliosarcoma cells, leukemia cells, or non-small cell lung cancer cells. In some aspects, the target site comprises one or more cancer cells of any of any cancer.

In some aspects, the subject has a cancer that is also resistant to PARP inhibitor treatment. In some aspects, it is determined that the cancer is also resistant to PARP inhibitor treatment prior to applying an alternating electric field. In some aspects, a cancer resistant to a PARP inhibitor can be a cancer that has progressed while on or after treatment with a PARP inhibitor.

In some aspects, the cancer has progressed on or after treatment with a PARP inhibitor. In some aspects, the cancer has progressed less than one month after stopping treatment with a PARP inhibitor, between one and six months after stopping treatment with a PARP inhibitor, between six months and 12 months after stopping treatment with a PARP inhibitor, or more than 12 months after stopping treatment with a PARP inhibitor.

In some aspects, the cancer is resistant to platinum-based systemic therapy and PARP inhibitor treatment. For example, the cancer can be resistant to carboplatin and olaparib.

In some aspects, the disclosed methods further comprise a step of administering a therapeutically effective amount of a PARP inhibitor to a subject. In some aspects, the PARP inhibitor can be administered prior to, after, or simultaneously with the alternating electric field. In some aspects, the PARP inhibitor can be administered prior to, after, or simultaneously with the platinum-based systemic therapy.

In some aspects, the PARP inhibitor is olaparib, niraparib, talazoparib, rucaparib, nesupariub, AZ3391, E7016, UPF 1069, AZ9482, AZD-2461, BYK204165, talazoparib, KCL-440, veliparib, CEP-9722, venadaparib, PJ34, stenoparib, amelparib, WD2000-012547, A-966492, DPQ, AG14361, NMS-P515, senaparib, simmiparib, mefuparib, OUL245, lerzeparib, BGP-15, YCH1899, basroparib, iniparib, 5-AIQ and NU1025.

In some aspects, a cancer resistant to PARP inhibitor treatment can be a cancer that is homologous recombination proficient (HRP), such as a cancer with wild type BRCA, that can repair the damage created by a PARP inhibitor. In some aspects, the cancer is homologous recombination deficient (HRD). In some aspects, a cancer that is HRD is a cancer that has a mutant BRCA gene.

In some aspects of any of the disclosed methods of treating, the alternating electric field is applied before, after, or simultaneously with administering the platinum-based systemic therapy. In some aspects of any of the disclosed methods of treating, the alternating electric field is applied before, after, or simultaneously with administering the PARP inhibitor.

In some aspects of any of the disclosed methods of treating, the frequency of the alternating electric field can be any of those disclosed herein. In some aspects, the frequency of the alternating electric field is between 100 kHz and 1 MHz. In some aspects, the frequency of the alternating electric field is about 150 kHz or about 200 kHz.

In some aspects of any of the disclosed methods of treating, the alternating electric field has a field strength of any of those disclosed herein. In some aspects, the alternating electric field has a field strength of between 0.5 and 4 V/cm RMS. In some aspects, the alternating electric field has a field strength of about 1 V/cm RMS.

In some aspects, the enhanced effect of the platinum-based therapy is compared to the platinum-based therapy in the absence of an alternating electric field.

### 2. Methods of Enhancing PARP Inhibitor Treatment

Disclosed are methods of enhancing the effect of a PARP inhibitor treatment in a subject having cancer comprising applying an alternating electric field to a target site of the subject for a period of time, the alternating electric field having a frequency and field strength, wherein the target site comprises one or more cancer cells, and administering a therapeutically effective amount of a PARP inhibitor to the subject, wherein the subject is resistant to platinum-based systemic therapy. In some aspects, a therapeutically effective amount of a PARP inhibitor when administered together with application of an alternating electric field would not have a therapeutic benefit in this patient population when the same amount of PARP inhibitor is administered without an alternating electric field.

In some aspects, a subject resistant to platinum-based systemic therapy can be a subject having a cancer that has progressed while on or after treatment with a platinum-based systemic therapy. In some aspects, a cancer resistant to platinum-based systemic therapy can be a cancer that never responded to a platinum-based systemic therapy. In some aspects, a cancer resistant to platinum-based systemic therapy can be a cancer that never responded to a platinum-based systemic therapy.

In some aspects, the cancer has progressed on or after treatment with a platinum-based systemic therapy. In some aspects, the cancer has progressed less than one month after stopping treatment with a platinum-based systemic therapy, between one and six months after stopping treatment with a platinum-based systemic therapy, between six months and 12 months after stopping treatment with a platinum-based systemic therapy, or more than 12 months after stopping treatment with a platinum-based systemic therapy.

Disclosed are methods of enhancing the effect of a PARP inhibitor treatment in a subject having cancer comprising applying an alternating electric field to a target site of the subject for a period of time, the alternating electric field having a frequency and field strength, wherein the target site comprises one or more cancer cells, and administering a therapeutically effective amount of a PARP inhibitor to the subject, wherein the subject is resistant to a PARP inhibitor. Accordingly, a therapeutically effective amount of a PARP inhibitor when administered together with application of an alternating electric field would not have a therapeutic benefit in this patient population when the same amount of PARP inhibitor is administered without an alternating electric field.

In some aspects, the subject has been diagnosed with radiological progression while on or after platinum-based systemic therapy.

In some aspects, the platinum-based systemic therapy can be, but is not limited to, one or more of nedaplatin, satraplatin, cisplatin, carboplatin, oxaliplatin, phenanthriplatin, picoplatin, and triplatin tetranitrate. Thus, in some aspects, a subject resistant to platinum-based systemic therapy is resistant to one or more of nedaplatin, satraplatin, cisplatin, carboplatin, oxaliplatin, phenanthriplatin, picoplatin, and triplatin tetranitrate.

In some aspects, the subject having cancer has ovarian cancer, breast cancer, pancreatic cancer, cervical cancer, brain cancer, hepatobiliary cancer, prostate cancer, head and neck cancers, glioblastoma, gliosarcoma, leukemia, or non-small cell lung cancer. In some aspects, the cancer can be, but is not limited to, ovarian cancer, hepatobiliary cancer, prostate cancer, pancreatic cancer, cervical cancer, head and neck cancers, glioblastoma, gliosarcoma, leukemia, or non-small cell lung cancer. Thus, in some aspects, the cancer cells are derived from one or more of these cancers.

In some aspects, the target site comprises one or more ovarian cancer cells. In some aspects, the target site comprises one or more breast cancer cells, pancreatic cancer cells, cervical cancer cells, brain cancer cells, hepatobiliary cancer cells, prostate cancer cells, head and neck cancer cells, glioblastoma cells, gliosarcoma cells, leukemia cells, or non-small cell lung cancer cells. In some aspects, the target site comprises one or more cancer cells of any of any cancer.

In some aspects, the PARP inhibitor is olaparib, niraparib, talazoparib, rucaparib, nesupariub, AZ3391, E7016, UPF 1069, AZ9482, AZD-2461, BYK204165, talazoparib, KCL-440, veliparib, CEP-9722, venadaparib, PJ34, stenoparib, amelparib, WD2000-012547, A-966492, DPQ, AG14361, NMS-P515, senaparib, simmiparib, mefuparib, OUL245, lerzeparib, BGP-15, YCH1899, basroparib, iniparib, 5-AIQ and NU1025. In some aspects, the PARP inhibitor is administered before, after, or simultaneously with applying the alternating electric field.

In some aspects, the cancer is also resistant to PARP inhibitor treatment. In some aspects, it is determined that the cancer is also resistant to PARP inhibitor treatment prior to applying an alternating electric field. In some aspects, a cancer resistant to a PARP inhibitor can be a cancer that has progressed while on or after treatment with a PARP inhibitor. In some aspects, a cancer resistant to a PARP inhibitor can be a cancer that never responded to a PARP inhibitor. In some aspects, a cancer resistant to a PARP inhibitor can be a cancer that never responded to a PARP inhibitor.

In some aspects, the cancer has progressed on or after treatment with a PARP inhibitor. In some aspects, the cancer has progressed less than one month after stopping treatment with a PARP inhibitor, between one and six months after stopping treatment with a PARP inhibitor, between six months and 12 months after stopping treatment with a PARP inhibitor, or more than 12 months after stopping treatment with a PARP inhibitor.

In some aspects, the cancer is resistant to both platinum-based systemic therapy and PARP inhibitor treatment. For example, the cancer can be resistant to carboplatin and olaparib treatment.

In some aspects, the disclosed methods further comprise a step of administering a therapeutically effective amount of a platinum-based systemic therapy to the subject. In some aspects, the platinum-based systemic therapy can be, but is not limited to, one or more of nedaplatin, satraplatin, cisplatin, carboplatin, oxaliplatin, phenanthriplatin, picoplatin, or triplatin tetranitrate. Thus, in some aspects, a cancer resistant to platinum-based systemic therapy is resistant to one or more of nedaplatin, satraplatin, cisplatin, carboplatin, oxaliplatin, phenanthriplatin, picoplatin, or triplatin tetranitrate. In some aspects, the platinum-based systemic therapy is administered before, after, or simultaneously with applying the alternating electric field and/or administering the PARP inhibitor.

In some aspects, the cancer is resistant to PARP inhibitor treatment. In some aspects, a cancer resistant to a PARP inhibitor treatment can be a cancer that is homologous recombination proficient (HRP), such as a cancer with wild type BRCA, that can repair the damage created by a PARP inhibitor.

In some aspects, applying an alternating electric field to a target site (e.g., tumor site or cancer cell) can sensitize the cancer to a PARP inhibitor treatment. In some aspects, sensitizing can include enhancing. A PARP inhibitor can prevent repair of double strand breaks in the DNA of cells, which, if not repaired, can lead to cell death. In some aspects, BRCA is known to play a role in DNA repair, therefore, in cells that BRCA (or the FANC/BRCA pathway) is not functional (e.g. mutant BRCA), the capacity of the cell to repair DNA damage is greatly reduced causing an accumulation of damaged DNA and cell death. In some aspects, a PARP inhibitor can further prevent DNA damage repair in BRCA mutant cells which cannot repair the damage and the cells die. In some aspects, a PARP inhibitor can be an effective treatment to remove unwanted cells, such as cancer cells. In some aspects, however, in wild type BRCA cells, a PARP inhibitor cannot be used as an effective treatment because the BRCA can help repair the DNA damage that the PARP inhibitor prevents PARP from repairing. In some aspects, applying an alternating electric field can downregulate multiple members of the FANC/BRCA pathway (not only BRCA), and the downregulation of just one member in this family can be enough to impair the function of the pathway. Thus, in some aspects, applying an alternating electric field can sensitize wild type BRCA cells to a PARP inhibitor treatment, thus allowing enhancement of treatment with a PARP inhibitor.

In some aspects of any of the disclosed methods of treating, the cancer is homologous recombination proficient (HRP). In some aspects, if a cancer is HRP, the subject is not administered a PARP inhibitor as a treatment. In some aspects, after applying an alternating electric field, a subject that has a cancer that is HRP can be administered a PARP inhibitor. In some aspects, a subject that has a cancer that is HRP is a subject that has a cancer with a wild type BRCA gene. In some aspects, if a subject has cancer that has a wild type BRCA gene, the subject is not administered a PARP inhibitor as a treatment. In some aspects, after applying an alternating electric field, subjects having a cancer having a wild type BRCA gene can be administered a PARP inhibitor.

In some aspects of any of the disclosed methods of treating, the cancer is homologous recombination deficient (HRD). In some aspects, a cancer that is HRD is a subject that has a mutant BRCA gene.

In some aspects, the alternating electric field is applied before, after, or simultaneously with administering the PARP inhibitor and/or platinum-based systemic therapy.

In some aspects of any of the disclosed methods of treating, the frequency of the alternating electric field can be any of those disclosed herein. In some aspects, the frequency of the alternating electric field is between 100 kHz and 1 MHz. In some aspects, the frequency of the alternating electric field is about 150 kHz or about 200 kHz.

In some aspects of any of the disclosed methods of treating, the alternating electric field has a field strength of any of those disclosed herein. In some aspects, the alternating electric field has a field strength of between 0.5 and 4 V/cm RMS. In some aspects, the alternating electric field has a field strength of about 1 V/cm RMS.

In some aspects, the enhanced effect of the PARP inhibitor is compared to the PARP inhibitor in the absence of an alternating electric field.

### 3. Methods of Treating

Disclosed herein is the use of an alternating electric field and a platinum-based systemic therapy and/or a PARP inhibitor to treat cancer.

Disclosed are methods of treating a subject having cancer comprising applying an alternating electric field to a target site of the subject for a period of time, the alternating electric field having a frequency and field strength, wherein the target site comprises one or more cancer cells, and administering a therapeutically effective amount of a platinum-based systemic therapy to the subject, wherein the cancer is resistant to platinum-based systemic therapy. In some aspects, a therapeutically effective amount of a platinum-based systemic therapy when administered together with application of an alternating electric field would not have a therapeutic benefit in this patient population when the same amount of platinum-based systemic therapy is administered without an alternating electric field.

In some aspects, the disclosed methods of treating comprise applying an alternating electric field to a target site of the subject for a period of time, the alternating electric field having a frequency and field strength, wherein the target site comprises one or more cancer cells, administering a therapeutically effective amount of a platinum-based systemic therapy to the subject, wherein the cancer is resistant to platinum-based systemic therapy, and further comprising administering a PARP inhibitor to the subject. In some aspects, a therapeutically effective amount of a platinum-based systemic therapy when administered together with application of an alternating electric field would not have a therapeutic benefit in this patient population when the same amount of platinum-based systemic therapy is administered without an alternating electric field.

Disclosed are methods of treating a subject having cancer comprising applying an alternating electric field to a target site of the subject for a period of time, the alternating electric field having a frequency and field strength, wherein the target site comprises one or more cancer cells, and administering a therapeutically effective amount of a PARP inhibitor to the subject. In some aspects, the cancer is resistant to platinum-based systemic therapy. In some aspects, the cancer is resistant to PARP inhibitor. In some aspects, the cancer is resistant to platinum-based systemic therapy and PARP inhibitor. In some aspects, a therapeutically effective amount of a platinum-based systemic therapy or PARP inhibitor when administered together with application of an alternating electric field would not have a therapeutic benefit in this patient population when the same amount of platinum-based systemic therapy or PARP inhibitor is administered without an alternating electric field.

In some aspects, the disclosed methods of treating comprise applying an alternating electric field to a target site of the subject for a period of time, the alternating electric field having a frequency and field strength, wherein the target site comprises one or more cancer cells, and administering a therapeutically effective amount of a PARP inhibitor to the subject, wherein the cancer is resistant to platinum-based systemic therapy, and further comprising administering a platinum-based systemic therapy to the subject. In some aspects, a therapeutically effective amount of a PARP inhibitor when administered together with application of an alternating electric field would not have a therapeutic benefit in this patient population when the same amount of PARP inhibitor is administered without an alternating electric field.

In some aspects of any of the disclosed methods of treating, the subject having cancer has ovarian cancer, breast cancer, pancreatic cancer, cervical cancer, brain cancer, hepatobiliary cancer, prostate cancer, head and neck cancers, glioblastoma, gliosarcoma, leukemia, or non-small cell lung cancer. In some aspects, the cancer can be, but is not limited to, ovarian cancer, hepatobiliary cancer, cervical cancer, prostate cancer, pancreatic cancer, head and neck cancers, glioblastoma, gliosarcoma, leukemia, or non-small cell lung cancer. Thus, in some aspects, the cancer cells are derived from one or more of these cancers.

In some aspects of any of the disclosed methods of treating, the target site comprises one or more ovarian cancer cells. In some aspects, the target site comprises one or more breast cancer cells, pancreatic cancer cells, cervical cancer cells, brain cancer cells, hepatobiliary cancer cells, prostate cancer cells, head and neck cancer cells, glioblastoma cells, gliosarcoma cells, leukemia cells, or non-small cell lung cancer cells. In some aspects, the target site comprises one or more cancer cells of any of any cancer.

In some aspects of any of the disclosed methods of treating, the platinum-based systemic therapy can be, but is not limited to, one or more of nedaplatin, satraplatin, cisplatin, carboplatin, oxaliplatin, phenanthriplatin, picoplatin, and triplatin tetranitrate. Thus, in some aspects, a subject resistant to platinum-based systemic therapy is resistant to one or more of nedaplatin, satraplatin, cisplatin, carboplatin, oxaliplatin, phenanthriplatin, picoplatin, and triplatin tetranitrate.

In some aspects of any of the disclosed methods of treating, apoptosis of cancer cells in the target site of the subject is increased or cell proliferation of cancer cells in the target site of the subject is decreased.

In some aspects of any of the disclosed methods of treating, the subject has a cancer that has previously been determined to be resistant to platinum-based systemic therapy prior to applying an alternating electric field. Thus, in some aspects, the alternating electric field allows for treatment of subjects with platinum-based systemic therapy having a cancer that was previously resistant to platinum-based systemic therapy.

In some aspects of any of the disclosed methods of treating, subjects resistant to platinum-based systemic therapy can be a cancer that has progressed while on or after treatment with a platinum-based systemic therapy. In some aspects, a cancer resistant to platinum-based systemic therapy can be a cancer that never responded to a platinum-based systemic therapy. In some aspects, a cancer resistant to platinum-based systemic therapy can be a cancer that never responded to a platinum-based systemic therapy.

In some aspects of any of the disclosed methods of treating, the subject has a cancer that has progressed on or after treatment with a platinum-based systemic therapy. In some aspects, the cancer has progressed less than one month after stopping treatment with a platinum-based systemic therapy, between one and six months after stopping treatment with a platinum-based systemic therapy, between six months and 12 months after stopping treatment with a platinum-based systemic therapy, or more than 12 months after stopping treatment with a platinum-based systemic therapy.

In some aspects of the disclosed methods of treating, the cancer is resistant to PARP inhibitor treatment. In some aspects, a cancer resistant to a PARP inhibitor can be a cancer that has progressed while on or after treatment with a PARP inhibitor. In some aspects, a cancer resistant to a PARP inhibitor can be a cancer that never responded to a PARP inhibitor.

In some aspects of any of the disclosed methods of treating, the cancer has previously been determined to be resistant to treatment with a PARP inhibitor prior to applying an alternating electric field. Thus, in some aspects, the alternating electric field allows for treatment of cancer with PARP inhibitor that were previously resistant to PARP inhibitor.

In some aspects, the cancer has progressed on or after treatment with a PARP inhibitor. In some aspects, the cancer has progressed less than one month after stopping treatment with a PARP inhibitor, between one and six months after stopping treatment with a PARP inhibitor, between six months and 12 months after stopping treatment with a PARP inhibitor, or more than 12 months after stopping treatment with a PARP inhibitor.

In some aspects, a cancer resistant to PARP inhibitor treatment can be a cancer that is homologous recombination proficient (HRP), such as a cancer with wild type BRCA, that can repair the damage prevented from being repaired by PARP by a PARP inhibitor.

In some aspects, applying an alternating electric field to a target site (e.g., tumor site or cancer cell) can sensitize the cancer to a PARP inhibitor treatment. In some aspects, sensitizing can include enhancing. A PARP inhibitor can prevent repair of double strand breaks in the DNA of cells, which, if not repaired, can lead to cell death. In some aspects, BRCA is known to play a role in DNA repair, therefore, in cells that BRCA (or the FANC/BRCA pathway) is not functional (e.g. mutant BRCA), the capacity of the cell to repair DNA damage is greatly reduced causing an accumulation of damaged DNA and cell death. In some aspects, a PARP inhibitor can further prevent DNA damage repair in BRCA mutant cells which cannot repair the damage and the cells die. In some aspects, a PARP inhibitor can be an effective treatment to remove unwanted cells, such as cancer cells. In some aspects, however, in wild type BRCA cells, a PARP inhibitor cannot be used as an effective treatment because the BRCA can help repair the DNA damage that the PARP inhibitor prevents PARP from repairing. In some aspects, applying an alternating electric field can downregulate multiple members of the FANC/BRCA pathway (not only BRCA), and the downregulation of just one member in this family can be enough to impair the function of the pathway. Thus, in some aspects, applying an alternating electric field can sensitize wild type BRCA cells to a PARP inhibitor treatment, thus allowing enhancement of treatment with a PARP inhibitor.

In some aspects of any of the disclosed methods of treating, the cancer is homologous recombination proficient (HRP). In some aspects, if a cancer is HRP, the subject is not administered a PARP inhibitor as a treatment. In some aspects, after applying an alternating electric field, a subject that has a cancer that is HRP can be administered a PARP inhibitor. In some aspects, a subject that has a cancer that is HRP is a subject that has a cancer with a wild type BRCA gene. In some aspects, if a subject has cancer that has a wild type BRCA gene, the subject is not administered a PARP inhibitor as a treatment. In some aspects, after applying an alternating electric field, subjects having a cancer having a wild type BRCA gene can be administered a PARP inhibitor.

In some aspects of any of the disclosed methods of treating, the cancer is homologous recombination deficient (HRD). In some aspects, a cancer that is HRD is a subject that has a mutant BRCA gene.

In some aspects of any of the disclosed methods of treating, the alternating electric field is applied before, after, or simultaneously with administering the platinum-based systemic therapy.

In some aspects of any of the disclosed methods of treating, the alternating electric field is applied before, after, or simultaneously with administering the PARP inhibitor.

In some aspects of any of the disclosed methods of treating, the frequency of the alternating electric field can be any of those disclosed herein. In some aspects, the frequency of the alternating electric field is between 100 kHz and 1 MHz. In some aspects, the frequency of the alternating electric field is about 150 kHz or about 200 kHz.

In some aspects of any of the disclosed methods of treating, the alternating electric field has a field strength of any of those disclosed herein. In some aspects, the alternating electric field has a field strength of between 0.5 and 4 V/cm RMS. In some aspects, the alternating electric field has a field strength of 1 about V/cm RMS.

### 4. Methods of Increasing Apoptosis

Disclosed are methods of increasing apoptosis in a cell comprising applying an alternating electric field to a cancer cell for a period of time, the alternating electric field having a frequency and field strength, and administering a therapeutically effective amount of a platinum-based systemic therapy to the cell, wherein the cell is resistant to platinum-based systemic therapy.

Also disclosed are methods of increasing apoptosis comprise applying an alternating electric field to a cell for a period of time, the alternating electric field having a frequency and field strength, wherein the cell is a cancer cell, contacting a therapeutically effective amount of a platinum-based systemic therapy to the cell, wherein the cell is resistant to platinum-based systemic therapy, and further comprising administering a PARP inhibitor to the cell.

Disclosed are methods of increasing apoptosis in a cell comprising applying an alternating electric field to a cell for a period of time, the alternating electric field having a frequency and field strength, and administering a therapeutically effective amount of a PARP inhibitor to the cell. In some aspects, the cell is resistant to platinum-based systemic therapy. In some aspects, the cell is resistant to a PARP inhibitor.

Also disclosed are methods of increasing apoptosis in a cell comprising applying an alternating electric field to a cell for a period of time, the alternating electric field having a frequency and field strength, and administering a therapeutically effective amount of a PARP inhibitor to the cell, wherein the cell is resistant to platinum-based systemic therapy, and further comprising administering a platinum-based systemic therapy to the subject.

Also disclosed are methods of increasing apoptosis in a cell comprising applying an alternating electric field to a cell for a period of time, the alternating electric field having a frequency and field strength, and administering a therapeutically effective amount of a PARP inhibitor to the cell, wherein the cell is resistant to a PARP inhibitor, and further comprising administering a platinum-based systemic therapy to the subject.

In some aspects, a therapeutically effective amount pf platinum-based systemic therapy when administered together with application of an alternating electric field would not have a therapeutic benefit in this patient population when the same amount of platinum-based systemic therapy is administered without an alternating electric field

In some aspects, the disclosed methods of increasing apoptosis occur in vitro. In some aspects, the disclosed methods occur in vivo. Thus, in some aspects, the cell is in a subject and therefore the alternating electric field and platinum-based systemic therapy and/or PARP inhibitor are applied or administered to the subject (e.g. indirectly to the cell), specifically a target site of the subject. For example, in some aspects, an in vivo method of increasing apoptosis comprises applying an alternating electric field to a target site of the subject for a period of time, the alternating electric field having a frequency and field strength, wherein the target site comprises one or more cancer cells, administering a therapeutically effective amount of a platinum-based systemic therapy to the subject, wherein the one or more cancer cells are resistant to platinum-based systemic therapy.

In some aspects of the disclosed methods of increasing apoptosis, the cell and/or cancer is further resistant to PARP inhibitor treatment.

In some aspects, the cancer can be, but is not limited to, ovarian cancer, hepatobiliary cancer, prostate cancer, pancreatic cancer, cervical cancer, head and neck cancers, glioblastoma, gliosarcoma, leukemia, or non-small cell lung cancer. Thus, in some aspects, the cancer cells are derived from one or more of these cancers. In some aspects, the subject having cancer can be a subject having, but is not limited to, ovarian cancer, breast cancer, pancreatic cancer, cervical cancer, brain cancer, hepatobiliary cancer, prostate cancer, head and neck cancers, glioblastoma, gliosarcoma, leukemia, or non-small cell lung cancer.

In some aspects of any of the disclosed methods of increasing apoptosis, the subject having cancer has ovarian cancer, breast cancer, pancreatic cancer, cervical cancer, brain cancer, hepatobiliary cancer, prostate cancer, head and neck cancers, glioblastoma, gliosarcoma, leukemia, or non-small cell lung cancer.

In some aspects, the target site comprises one or more ovarian cancer cells. In some aspects, the target site comprises one or more breast cancer cells, pancreatic cancer cells, cervical cancer cells, brain cancer cells, hepatobiliary cancer cells, prostate cancer cells, head and neck cancer cells, glioblastoma cells, gliosarcoma cells, leukemia cells, or non-small cell lung cancer cells. In some aspects, the target site comprises one or more cancer cells of any of any cancer.

In some aspects of any of the disclosed methods of increasing apoptosis, the platinum-based systemic therapy can be, but is not limited to, nedaplatin, satraplatin, cisplatin, carboplatin, oxaliplatin, phenanthriplatin, picoplatin, or triplatin tetranitrate. Thus, in some aspects, a subject resistant to platinum-based systemic therapy is resistant to nedaplatin, satraplatin, cisplatin, carboplatin, oxaliplatin, phenanthriplatin, picoplatin, or triplatin tetranitrate.

In some aspects of any of the disclosed methods of increasing apoptosis, apoptosis of cancer cells in the target site of the subject is increased.

In some aspects of any of the disclosed methods of increasing apoptosis, the cell or the cancer has previously been determined to be resistant to platinum-based systemic therapy prior to applying an alternating electric field. Thus, in some aspects, the alternating electric field allows for treatment of cells or cancers with platinum-based systemic therapy that were previously resistant to platinum-based systemic therapy.

In some aspects of any of the disclosed methods of increasing apoptosis, cells or cancers resistant to platinum-based systemic therapy can be a cancer or a cancer cell from a tumor that has progressed while on or after treatment with a platinum-based systemic therapy. In some aspects, a cell or cancer resistant to platinum-based systemic therapy can be a cell or cancer that never responded to a platinum-based systemic therapy.

In some aspects of any of the disclosed methods of increasing apoptosis, the cell or cancer has progressed on or after treatment with a platinum-based systemic therapy. In some aspects, the cancer has progressed less than one month after stopping treatment with a platinum-based systemic therapy, between one and six months after stopping treatment with a platinum-based systemic therapy, between six months and 12 months after stopping treatment with a platinum-based systemic therapy, or more than 12 months after stopping treatment with a platinum-based systemic therapy.

In some aspects of the disclosed methods of increasing apoptosis, the cell or cancer resistant to platinum-based systemic therapy is further resistant to PARP inhibitor treatment. In some aspects, a cell or cancer resistant to a treatment with a PARP inhibitor can be a cancer or a cancer cell from a tumor that has progressed while on or after treatment with a PARP inhibitor. In some aspects, a cancer resistant to a PARP inhibitor can be a a cancer that never responded to treatment with a PARP inhibitor.

In some aspects, the subject having cancer has progressed on or after treatment with a PARP inhibitor. In some aspects, the cancer has progressed less than one month after stopping treatment with a a PARP inhibitor, between one and six months after stopping treatment with a a PARP inhibitor, between six months and 12 months after stopping treatment with a a PARP inhibitor, or more than 12 months after stopping treatment with a PARP inhibitor.

In some aspects, the in vivo methods described herein can be performed on any of the subjects disclosed throughout the specification.

### E. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising one or more of platinum-based systemic therapy and one or more materials for delivering alternating electric fields, such as the Optune system. For example disclosed are kits comprising one or more of nedaplatin, satraplatin, cisplatin, carboplatin, oxaliplatin, phenanthriplatin, picoplatin, or triplatin tetranitrate and one or more materials for delivering alternating electric fields, such as the Optune system. In some aspects, the kits can further comprise a PARP inhibitor

Disclosed are kits comprising one or more of PARP inhibitor and one or more materials for delivering alternating electric fields, such as the Optune system.

### EXAMPLES

### A. Example 1: Tumor Treating Fields (TTFields) Concomitant with PARP Inhibitors or Carboplatin for Treatment of Ovarian Cancer Cell Lines

### 1. Introduction

Platinum-based chemotherapy is recommended after surgery for most patients with ovarian cancer; and PARP inhibitors (PARPi) are recommended as maintenance therapy. Niraparib and olaparib are approved PARPi for first-line maintenance therapy for woman with advanced ovarian cancer; however, 3 out of 4 patients do not harbor BRCA mutations, and thus may experience limited benefit from PARP inhibition. Tumor Treating Fields (TTFields) are electric fields that exert physical forces to disrupt cellular processes critical for cancer cell viability and tumor progression and have been shown to induce a state of BRCAness in various cancer types. TTFields have shown in vitro and in vivo efficacy in ovarian cancer models, with an optimal frequency of 200 kHz.

The objective of this study was to examine in vitro the potential of concomitant use of TTFields with PARPi or carboplatin for ovarian cancer treatment.

### 2. Methods

TTFields application: Ovarian carcinoma cells A2780 (BRCAWT) and OVCAR3 (BRCA mutated) were treated for 72 h with TTFields at an intensity of 1 V/cm RMS and a frequency of 200 kHz using the inovitro system.

Co-application experiments: TTFields were applied to the cells for 72 h in the absence or presence of various concentrations of the PARP inhibitors olaparib or niraparib. Synergy was defined when the observed value was lower than that predicted for an additive effect (calculated by multiplying the observed effects for TTFields alone with that for olaparib/niraparib alone).

Cytotoxic effect: Treated cells were counted using a flow cytometer, and cytotoxic effect was calculated relative to control cells.

Overall effects: Treated cells were harvested, re-plated, and grown for an additional 7-14 days. Colonies were stained (0.5% crystal violet solution), counted, and clonogenic effect was calculated relative to control. Overall effect was calculated by multiplying the cytotoxic and clonogenic effects.

Apoptosis: Treated cells were double-stained with FITC-conjugated annexin V (AnnV) and 7-aminoactinomycin D (7AAD), and data acquisition was performed with a flow cytometer.

### 3. Conclusions

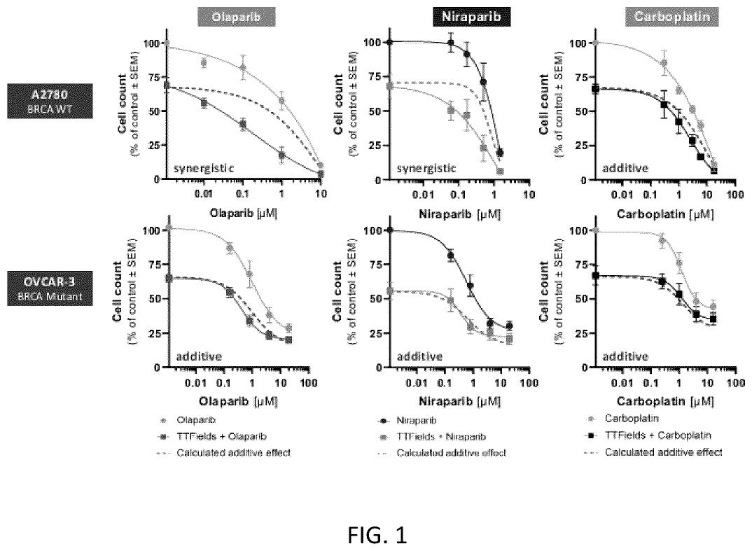

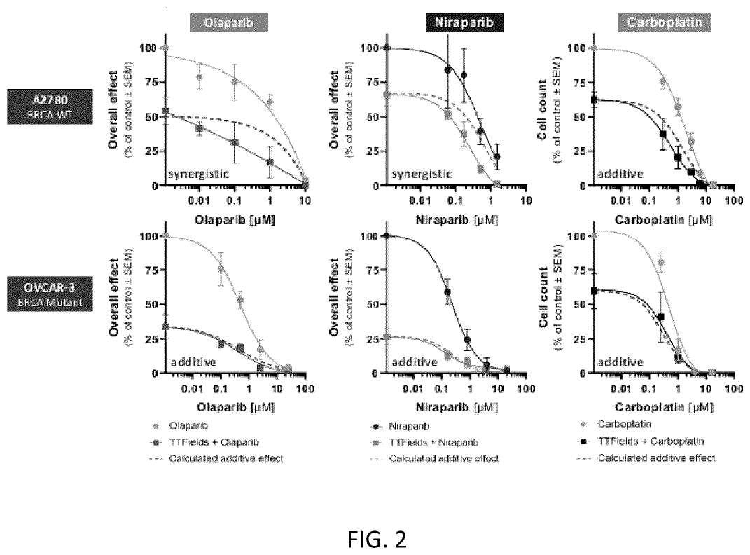

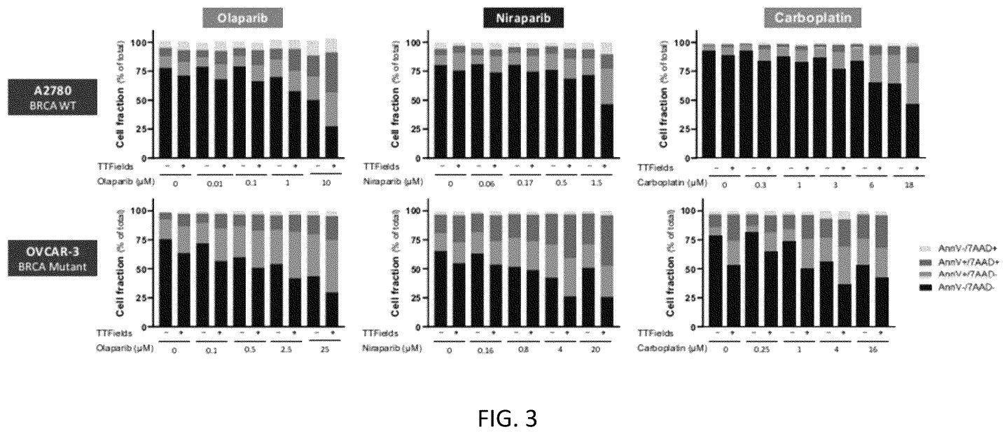

In the A2780 BRCA wild type cells, concomitant TTFields with PARPi displayed a synergistic interaction; and co-application with carboplatin showed a tendency to synergism (See FIGS. 1-3). In the OVCAR-3 BRCA mutant cells, application of TTFields together with PARPi or carboplatin yielded an additive effect. These outcomes are in line with the BRCAness state induced by TTFields, the known synthetic lethality of PARPi with BRCA deficiency, and the involvement of the FA-BRCA pathway in pair of DNA damage induced by these modalities.

The data suggest potential benefits for TTFields concomitant with platinum-based chemotherapy or PARPi in ovarian cancer, even in the absence of background BRCA mutations.

### B. Example 2: The Effect of Concomitant Treatment of TTFields and Carboplatin in Ovarian Carcinoma Cell Lines

### 1. Overview

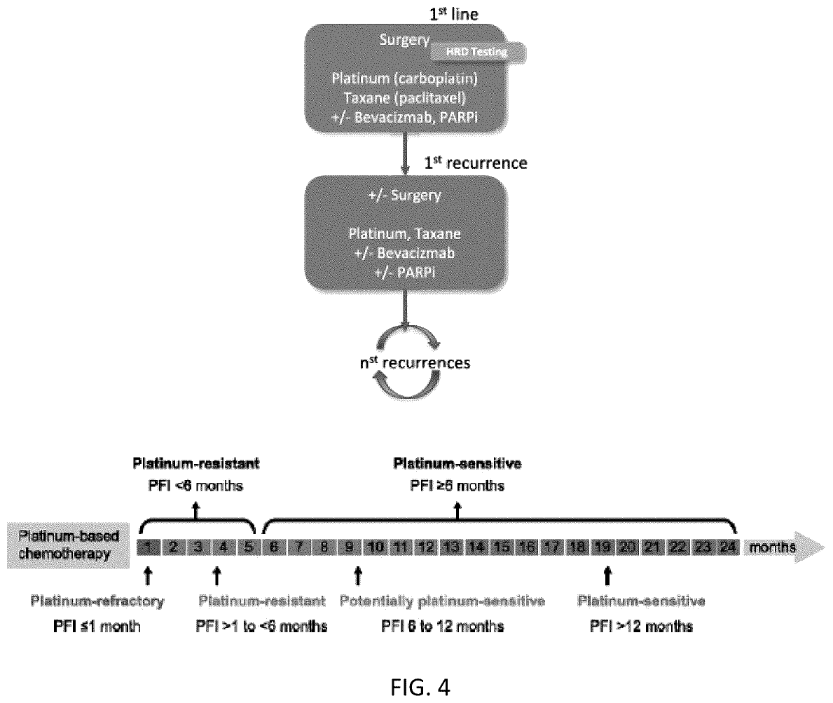

FIG. 4 shows a treatment paradigm for epithelial ovarian cancer. Approximately 80% of patients with ovarian cancer are treated with cytoreductive surgery followed by adjuvant chemotherapy with carboplatin and paclitaxel or cisplatin and paclitaxel. About 70% of patients with this treatment regimen will relapse and the recurring cancer is often resistant to standard platinum-based chemotherapy. Cross-resistance to platinum and PARPi can be present and more than two-thirds of patients on long-term PARPi therapy will eventually develop PARPi resistance.

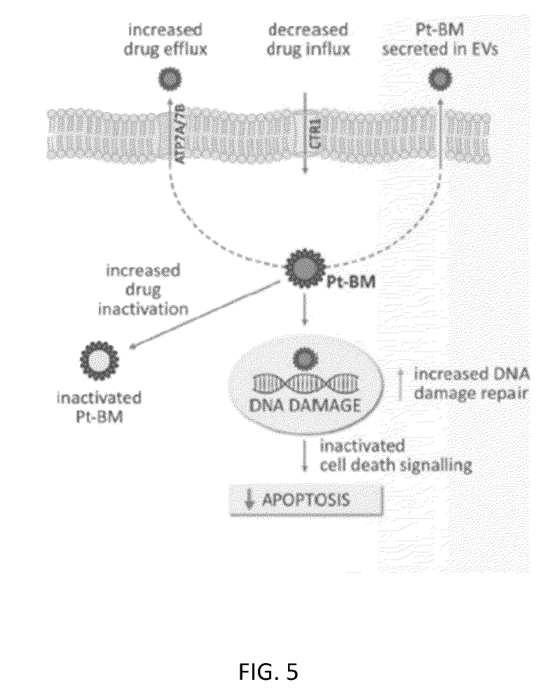

FIG. 5 shows examples of mechanisms of platinum resistance. FIG. 5 also shows reduced influx, increased efflux, reduced accumulation of Pt-based agents, increased activity of DNA damage repair processes, genetic mutations and epigenetic upregulation of DDR (DNA damage repair) proteins.

### 2. Methods and Results

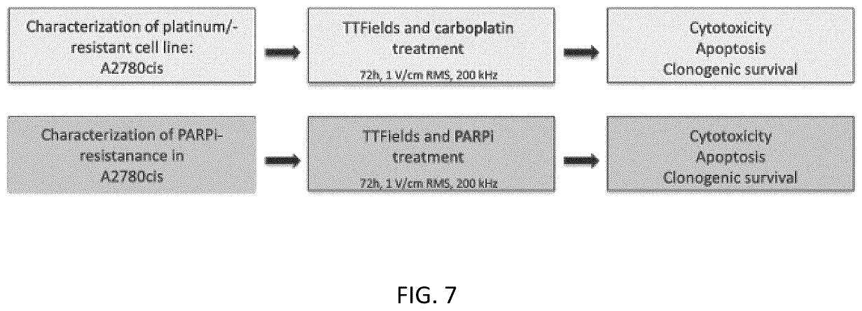

The study objectives are to evaluate the effect of concomitant treatment of TTFields and carboplatin on cytotoxicity, apoptosis and clonogenic survival in ovarian cancer cell lines (A2780, OVCAR3) and to study the effect of TTFields on cytotoxicity, apoptosis and clonogenic survival in a carboplatin-resistant ovarian cell line (see FIG. 7).

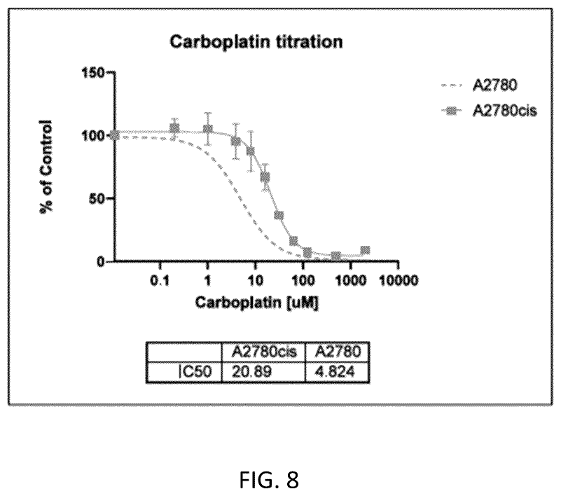

FIG. 8 shows the titration of carboplatin for A2780 wildtype cell line that is responsive to carboplatin (in dotted line) compared to A2780cis cell line that is resistant to carboplatin that exhibited lower response to carboplatin (solid line).

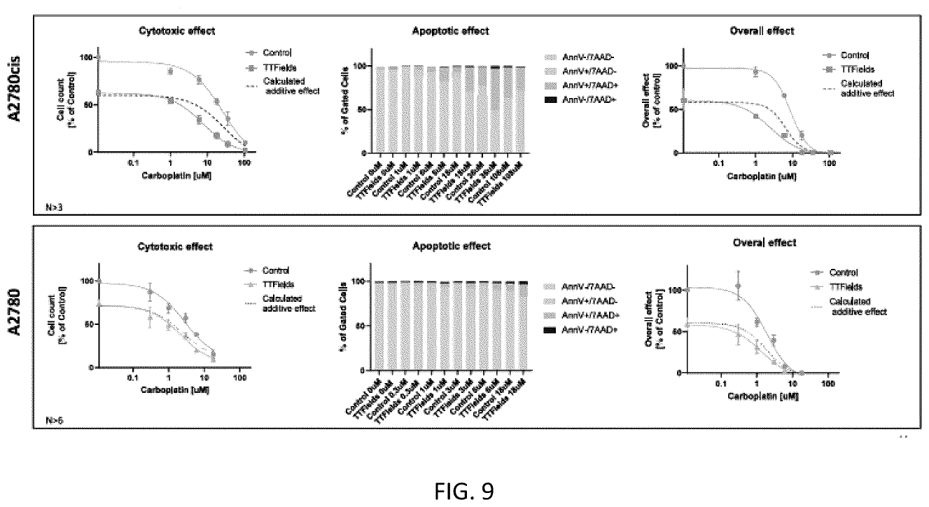

FIG. 9 shows the results of TTFields application on ovarian carcinoma cells A2780 and A2780cis (cisplatin resistant cells) treated for 72 h with TTFields at an intensity of 1 V/cm RMS and a frequency of 200 kHz using the INOVITRO system.

Co-application experiments are shown in FIG. 9. TTFields were applied to the cells for 72 h in the absence or presence of various concentrations of carboplatin. Synergy was defined when the observed value was lower than that predicted for an additive effect (presented by the red dotted line-calculated by multiplying the observed effects for TTFields alone with that for cisplatin alone).

The Cytotoxic effect was also determined. Treated cells were counted using a flow cytometer, and cytotoxic effect was calculated relative to control cells.

Treated cells were harvested, re-plated, and grown for an additional 7-14 days. Colonies were stained (0.5% crystal violet solution), counted, and clonogenic effect was calculated relative to control. The overall effect was calculated by multiplying the cytotoxic and clonogenic effects.

Apoptosis was also examined. Treated cells were double-stained with FITC-conjugated annexin V (AnnV) and 7-aminoactinomycin D (7AAD), and data acquisition was performed with a flow cytometer.

In the A2780 BRCA wild type cells, concomitant TTFields with carboplatin displayed an additive interaction, while treatment of TTFields with carboplatin in A2780Cis cells displayed a synergistic effect. The data shows the potential benefits for TTFields concomitant with platinum-based chemotherapy in ovarian cancer in cancers that are resistant to platinum based chemotherapies.

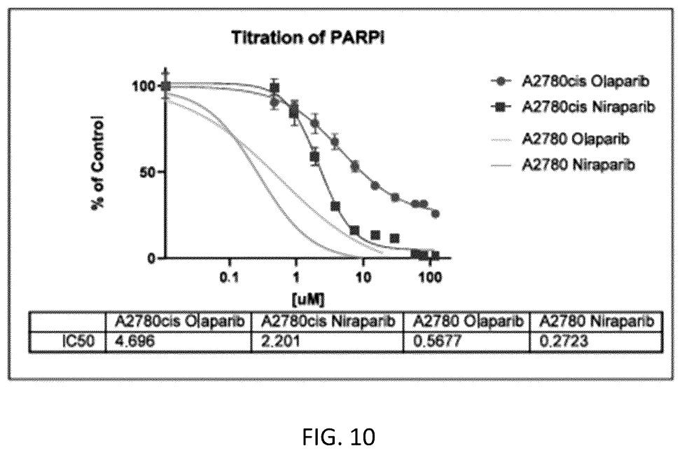

Titration of PARPi Olaparib and Niraparib for A2780 wildtype cell line that is responsive to carboplatin compared to A2780cis cell line that is resistant to carboplatin that exhibited lower response to carboplatin (See FIG. 10).

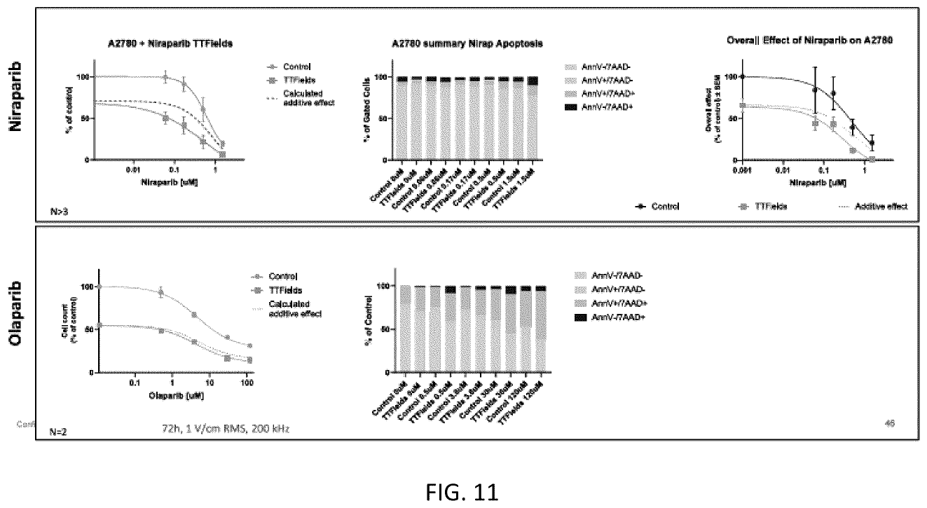

TTFields application was studied. Ovarian carcinoma cells A2780cis (cisplatin resistant cells) were treated for 72 h with TTFields at an intensity of 1 V/cm RMS and a frequency of 200 kHz using the inovitro system (See FIG. 11).

Co-application experiments are shown in FIG. 11. TTFields were applied to the cells for 72 h in the absence or presence of various concentrations of PARPi Olaparib and Niraparib. Synergy was defined when the observed value was lower than that predicted for an additive effect (presented by the red dotted line-calculated by multiplying the observed effects for TTFields alone with that for cisplatin alone).

The cytotoxic effect was studied. Treated cells were counted using a flow cytometer, and cytotoxic effect was calculated relative to control cells.

Treated cells were harvested, re-plated, and grown for an additional 7-14 days. Colonies were stained (0.5% crystal violet solution), counted, and clonogenic effect was calculated relative to control. Overall effect was calculated by multiplying the cytotoxic and clonogenic effects.

Treated cells were double-stained with FITC-conjugated annexin V (AnnV) and 7-aminoactinomycin D (7AAD), and data acquisition was performed with a flow cytometer.

### 3. Conclusions

TTFields increased the sensitivity of A2780cis platinum-resistant cell line to carboplatin and niraparib. The results show that TTFields enhances the effect of both carboplatin and niraparib in A2780cis. Taken together, the data shows that TTFields can increase sensitivity to both types of drugs through different mechanisms of action.

### C. Example 3: Tumor Treating Fields (TTFields) Induce Homologous Recombination Deficiency in Ovarian Cancer Cells, Thus Mitigating Drug Resistance

### 1. Introduction

Ovarian cancer has the worst prognosis among gynecological malignancies. First-line standard-of-care treatment includes debulking surgery in combination with either adjuvant or neo-adjuvant treatment with a platinum-taxane doublet, mainly carboplatin and paclitaxel. This can be supplemented with therapy consisting of an angiogenesis inhibitor (bevacizumab) and/or maintenance with a poly (ADP-ribose) polymerase inhibitor (PARPi), olaparib, niraparib or rucaparib. Despite promising initial responses to therapy, approximately 80% of women experience disease progression or recurrence.

Over the last two decades, it has become well established that germline mutations and epigenetic silencing of tumor suppressor genes can be associated with a significantly elevated risk of ovarian cancer development and a more aggressive disease. Inherited mutations in BRCA related genes impair the ability of cells to repair DNA double strand breaks (DSB) through homologous recombination (HR) and their ability to support replication fork stabilization, overall leading to replication stress and genomic instability. These mutations hence create a fertile ground for the accumulation of genetic alterations and an increased likelihood of uncontrolled cell proliferation, driving cancer development. Such genes include BRCA1, BRCA2, and other genes involved in the HR pathway, traits collectively referred to as “BRCAness”. Tumor cells possessing mutations in the HR pathway are also referred to as HR deficient (HRD) cells, as opposed to HR proficient (HRP) cells that exhibit normal expression patterns.

While individuals with HRD tumors are at elevated risk of malignant transformation, they also exhibit increased sensitivity to ovarian cancer therapies targeting DNA damage and repair mechanisms, such as platinum-based chemotherapy and PARPi, respectively. Platinum-based chemotherapy forms DNA inter- and intrastrand crosslinks, leading to stalled replication forks and consequent development of DSB. HRD cells present with conditional vulnerability to such chemotherapy drugs, due to their reduced damage repair capacity leading to accumulation of DNA damage, which can induce cell death.

The particular efficacy of PARPi in HRD patients is due to the concept known as synthetic lethality, in which the individual loss of either one of two genes involved in DNA damage repair can be viable, while their simultaneous loss of activity is fatal. Inhibition of PARP impairs base excision repair (BER) activity, limiting the cell's ability to repair single strand breaks (SSB), which when left unrepaired may develop into DSB. PARPi have further been suggested to trap the PARP enzyme within the DNA, resulting in replication fork collapse and consequent DSB formation. Accordingly, when HRD patients are treated with PARPi, synthetic lethality occurs due to deficiencies in both HR and BER pathways, the former related to the genetic predisposition of the cells, and the latter stemming from targeted inhibition by treatment with PARPi.

Ovarian cancer patients often present with therapy resistance after prior treatment. Several mechanisms have been suggested to explain the acquired tumor resistance to platinum-based and PARPi therapies, including dysregulation of drug influx and efflux. Of note, acquired resistance has also been suggested to involve restoration of HR function in HRD tumors, either through secondary mutations (somatic insertion/deletion that cause a frameshift that reinstates the open reading frame) or epigenetic modifications (loss of promoter hypermethylation). Because three out of four ovarian cancer patients are HRP, and since patients that were initially HRD may acquire treatment resistance via transformation to an HRP-like phenotype, therapies that impose BRCAness may facilitate synthetic lethality, potentially augmenting the efficacy of PARPi.

It has been shown that a state of BRCAness can be induced by Tumor Treating Fields (TTFields), a clinically approved antimitotic cancer treatment, in which electric fields are continuously and non-invasively applied to the tumor bed. Specifically, TTFields-induced downregulation of DNA repair proteins from the Fanconi Anemia (FA)-BRCA pathway has been preclinically demonstrated in several tumor types; and exploitation of this induced state of BRCAness to enhance the effects of olaparib has been shown in non-small cell lung carcinoma (NSCLC) models. In accordance with the involvement of the FA-BRCA pathway in the repair of DNA damage induced by platinum agents, TTFields have also been observed to augment the effect of cisplatin in preclinical models of pleural mesothelioma and NSCLC.

TTFields therapy is currently approved in the U.S., Canada, China, Hong Kong, Japan, Europe, Israel, and Australia for treatment of newly diagnosed glioblastoma (GBM) concomitant with the DNA alkylating agent temozolomide; and in the U.S., Israel, and Europe for treatment of pleural mesothelioma concomitant with the DNA damaging agents cisplatin and pemetrexed.

The research described herein examined the efficacy of TTFields co-treatment with ovarian cancer standard therapies, namely carboplatin, olaparib, and niraparib, which induce DNA damage or interfere with DNA damage repair, in order to sensitize the cells to treatment owing to the plausible HRD-like state induced by TTFields. The co-treatment demonstrating highest benefit in vitro was also tested in an ovarian cancer animal model.

### 2. Methods and Materials

### Cell Culture

The human ovarian endometrioid adenocarcinoma cell lines A2780 and A2780cis were obtained from the European Collection of Cell Cultures and from AddexBio, respectively. The human ovarian high grade ovarian serous adenocarcinoma cell line OVCAR-3 was obtained from the American Type Culture Collection (ATCC). Human cell lines were grown in RPMI media supplemented with 10% (v/v) fetal bovine serum (FBS), 1 mM sodium pyruvate, 12 mM HEPES and penicillin/streptomycin (50 μg/ml) in a 37° C. humidified incubator supplied with 5% CO₂. The media of A2780cis cells was additionally supplemented with 1 μM cisplatin (Sigma, C2210000) in every passage to maintain platinum resistance. Media and supplements were purchased from Sartorius Israel Ltd. (Biological Industries Ltd., Beit HaEmek). Murine ID8 cells were previously transduced by the Laboratory of Molecular Virology and Gene Therapy in the Leuven Viral Vector Core of KU Leuven, using a lentiviral vector (pCHMWS_CMV-fluc-I-PuroR) to create the stable luciferase producing cell line, ID8-fLuc (Baert et al., 2015). These ID8-fluc cells were cultured at 37° C. with 5% CO₂ in Dulbecco's Modified Eagle Medium (DMEM) supplemented with 10% FCS, 100 U/ml penicillin/streptomycin, 2 mM glutamine, 2.5 μg/ml amphotericin B and 10 mg/ml gemcitabine, which were obtained from Gibco.

### Application of TTFields to Cells

Cells were seeded on coverslip (22 mm diameter; 20×10³ cells/coverslip for A2780 and A2780cis; 40×10³ cells/coverslip for OVCAR-3). After overnight incubation, the coverslips were transferred into in vitro dishes containing 2 ml of media. TTFields at a frequency of 200 kHz (and intensity of 1 V/cm RMS) were applied to the cells for 72 hr using the INOVITRO system (Novocure, Haifa, Israel).

Co-Application of TTFields with Drugs to Cell Lines

For efficacy outcomes (cell count, overall effect, and apoptosis), various concentrations of carboplatin (MCE MedChemExpress, HY-17393), olaparib (Cayman Chemical, 10621), or niraparib (Cayman Chemical, 20842) were applied, with or without TTFields.

For DNA damage and cell cycle examination, the following drug concentrations were selected: For A2780—6 μM carboplatin, 1 μM olaparib, and 0.5 μM niraparib; For OVCAR-3-16 μM carboplatin, 0.5 μM olaparib, and 0.8 μM niraparib; For A2780cis—36 μM carboplatin, 10 μM olaparib, and 1.5 μM niraparib.

### Cell Count

Cell count was examined following treatment using Cytek Northern Lights flow cytometer (Cytek Biosciences, USA). Results are presented as a percentage relative to control.

### Overall Effect

Treated cells were harvested, re-plated in 6-well plates (500 cells/well for A2780 and A2780cis; 1000 for cells/well OVCAR-3), and grown for 7 (A2780 and A2780cis) or 21 (OVCAR-3) days. Colonies were stained with 0.5% crystal violet, quantified with ImageJ, and expressed as percentages relative to control. Overall effect was calculated by multiplying colony formation with the corresponding cell count.

### Determining the Type and Magnitude of TTFields-Drug Interactions

The surviving fraction predicted for an additive effect between TTFields and drug was calculated per the various drug concentrations by multiplying the actual measured surviving fractions for the individual treatments one by the other (SF_{calculated additive}=SF_{TTFields}×SF_{drug}; SF are expressed as probability). Based on the calculated values, a trendline was determined. Additivity, synergy, or antagonism was defined when the calculated additive trendline overlapped, was above, or was below the actual measured line for TTFields+drug, respectively.

For quantifying the magnitude of TTFields-drug interaction, interaction index (Iᵢ) values were calculated by the Bliss independence method using mortality values (Mₓ=1−SFₓ). Per the various drug concentrations, mortality predicted for an additive effect between TTFields and drug (M_{calculated additive}=M_{TTFields}+M_{drug}−M_{TTFields}×M_{drug}) was divided by the actual measured mortality for TTFields+drug. Additivity was determined when the 95% confidence interval (CI) overlapped 1, synergy when 95% CI<1, and antagonism when 95% CI>1. Lower Iᵢ values were considered indicative of higher synergy levels.

### Apoptosis

Treated cells were stained with FITC-conjugated Annexin V (AnnV) and 7-Aminoactinomycin D (7-AAD) using a commercial kit (BioLegend, San Diego, CA, USA), according to the manufacturer's instruction. Data acquisition and analysis were done on the Cytek Northern Lights flow cytometer

### Western Blot Analysis

Extracts were prepared from treated cells and subjected to western blot analysis (25 g protein/sample) as previously described. Primary antibodies are outlined in Table 1. Horseradish peroxidase (HRP)-conjugated secondary antibody (Abcam, Cambridge, UK; cat #ab97023 or #ab6721, 1:10,000) and a chemiluminescent substrate (Immobilon Forte, Millipore, Burlington, MA, USA) were used for visualization. Bands were recorded on GeneGnome XRQ gel imager (AlphMetrix Bitech, Rödermark, Germany). Densitometric readings were normalized to GAPDH with FIJI software and expressed as fold change relative to control.

### DNA Damage Examination

Treated cells were fixed with 4% paraformaldehyde for 10 min, permeabilized for 20 min with 0.5% Triton X-100 in PBS, and blocked with donkey serum (PBS with 0.3% Triton X-100 and donkey serum 1:100). Cells were incubated at 4° C. overnight with anti-γH2AX antibody (Cell Signaling, Danvers, MA, USA; #9718, 1:400), followed by incubation at room temperature for 1 hr with Alexa Flour 488-conjugated secondary antibody (Jackson Immunoresearch, Cambridge, UK; #711-545-152, 1:500) and 0.2 g/ml 4′,6-diamidino-2-phenylindole (DAPI; Sigma Aldrich, Rehovot, Israel). LSM 700 laser scanning confocal system (Zeiss, Gottingen, Germany) was utilized to obtain images, and the mean number of foci per nucleus was determined using the FIJI software with the BioVoxxel plugin.

### Cell Cycle Analysis

Treated cells were fixed with 70% ice-cold ethanol for 30 min, pelleted, washed, and stained for 30 min at 37° C. in phosphate buffered saline (PBS) containing 1% FBS 5 μg/ml 7-AAD (BioLegend), 200 μg/ml RNase, 1 mM EDTA and 0.1% Triton X-100. Data acquisition (at 665/30 nm) and analysis were done on the Cytek Northern Lights flow cytometer and the FlowJo 10.8.1 software (BD Biosciences), respectively.

### Co-Application of TTFields and Olaparib In Vivo

Murine experiments were approved by the KU Leuven ethical committee (P082/2021). NIH guidelines for the Care and Use of Laboratory Animals were followed along with the 2010/62/EU directive and the ARRIVE (Animal Research: Reporting of In Vivo Research: Reporting of In Vivo Experiments) guidelines. Syngeneic ID8-fluc cells were harvested using 0.05% Trypsin-EDTA and inoculated (5×10⁶ cells in 100 μL DPBS) intraperitoneally in female C57BL/6 mice (six- to eight-week-old, obtained from Envigo (Horst, The Netherlands)), leading to the development of a stage III-IV ovarian cancer model.

Seven days post inoculation, treatment was initiated. Mice were divided into four groups receiving either, sham-heat and vehicle (n=8), sham-heat and olaparib (n=8), TTFields and vehicle (n=7) or TTFields and olaparib (n=11). TTFields treatment (200 kHz) was administered continuously using the in vivo system (Novocure, Israel) by applying electrode arrays to the shaven abdomen of the mice as previously described. Sham-heat used analogous non-therapeutic arrays. Olaparib (MedKoo Biosciences, USA) was dissolved in a vehicle of 10% DMSO, 50% PEG300 and 40% DPBS and administered at a concentration of 50 mg/kg/day through daily oral gavage. Overall, therapeutic interventions lasted for four weeks, given in four cycles of five consecutive treatment days, followed by two days without treatment.

### Bioluminescent Imaging

Tumor load was observed through bioluminescent imaging analysis before (day 5) and after (day 35) treatment administration. All mice were anaesthetized using isoflurane gas (2 L/min) and received 126 mg/kg of D-luciferin through subcutaneous injection. Ten minutes post injection, photon flux was measured using the IVIS-spectrum preclinical In Vivo Imaging System (Perkin-Elmer, USA). Normalized photon flux was calculated by subtracting the photon flux before treatment from the paired photon flux after treatment per mouse.

### Overall Survival

Mice were followed up and weighed daily once ascites development started as indicated by the appearance of abdominal distention. Mice were sacrificed when their body weight reached ≥32 grams as a surrogate endpoint for survival.

### Statistical Analysis

In vitro experiments were repeated at least three times, and data are presented as mean±standard error of the mean (SEM), and analyzed with ANOVA or student's t-test as appropriate. To determine the in vivo sample size, a statistical power analysis was performed to reach a power of at least 0.80. Photon-flux measured through BLI was summarized with means and standard deviations and visualized using bar charts. These results were analyzed using one-way ANOVA with Tukey's multiple comparisons test. Kaplan-Meier curves were compared using the log-rank test. Multiple comparison adjustment was performed using the Benjamini-Hochberg procedure. Statistical analyses were performed using GraphPad Prism 10.1 software (La Jolla) and differences considered significant at (adjusted) p-values of: *p<0.05, **p<0.01, and ***p<0.001.

### 3. Results

TTFields Enhance the Efficacy of Carboplatin, Additively in HRD Cells and with a Tendency to Synergy in HRP and Platinum-Resistant Cells

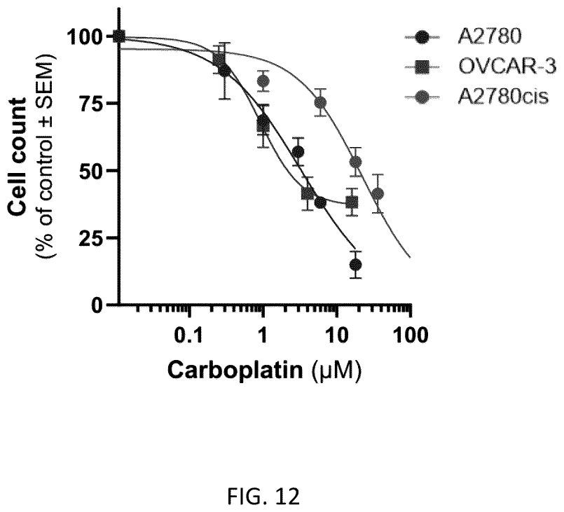

Carboplatin dose response curves were measured based on cell count measurements, in three different human ovarian cell lines: A2780 (HRP cells), OVCAR-3 (HRD cells), and A2780cis (platinum-resistant cells, commercially available generated by repeated exposures of the A2780 cell line to cisplatin). The OVCAR-3 cells demonstrated highest sensitivity to carboplatin, while the A2780cis cells demonstrated highest resistance, as would be expected by the HRD phenotype of the former and the acquired resistance of the latter (FIG. 12).

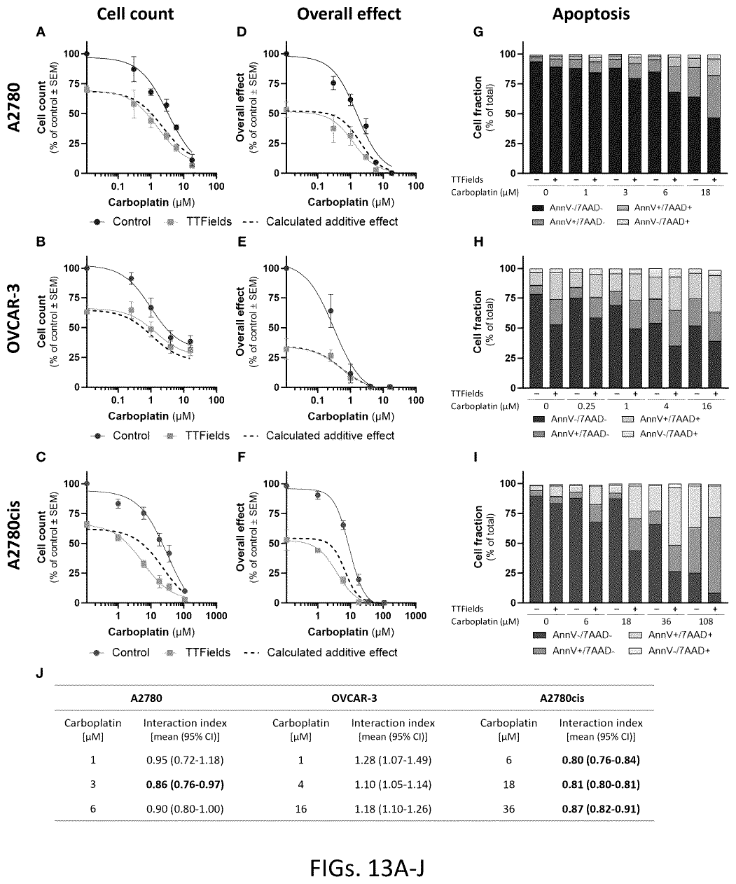

TTFields significantly amplified (i.e. lower cell count) the effect induced by carboplatin alone in all examined cell lines (FIGS. 13A-C; p<0.0001 for all cell lines). Similarly, the overall effect (cell count×colony formation) induced by carboplatin was significantly elevated after addition of TTFields (FIGS. 13D-F; p<0.0001 for all cell lines). Apoptosis analysis demonstrated increases in the apoptotic cell fraction when TTFields were applied with carboplatin, suggesting a cytotoxic effect (FIGS. 13G-I).

While the concomitant application of TTFields with carboplatin led to enhanced treatment efficacy relative to each treatment alone, the nature of interaction between the two modalities was further analyzed. The expected dose curve for an additive effect was calculated (FIGS. 13A-F, dashed lines) and the interaction index (Iᵢ, FIG. 13J). An antagonistic interaction was demonstrated in OVCAR-3 cells, as the actual measured curves were above the calculated additive curves for co-treatment and the 95% confidence intervals (CI) for Iᵢ were larger than 1. On the other hand, for A2780 and A2780cis cells synergy was determined, as the actual measured curves were below the calculated additive curves and the 95% CI for Iᵢ were smaller than 1. The lower Iᵢ determined for A2780cis relative to A2780 cells suggested higher levels of TTFields plus carboplatin synergy in A2780cis cells.

TTFields Enhance the Efficacy of PARPi, Additively in HRD Cells, with a Tendency to Synergy in Platinum-Resistant Cells, and with High Synergy in HRP Cells

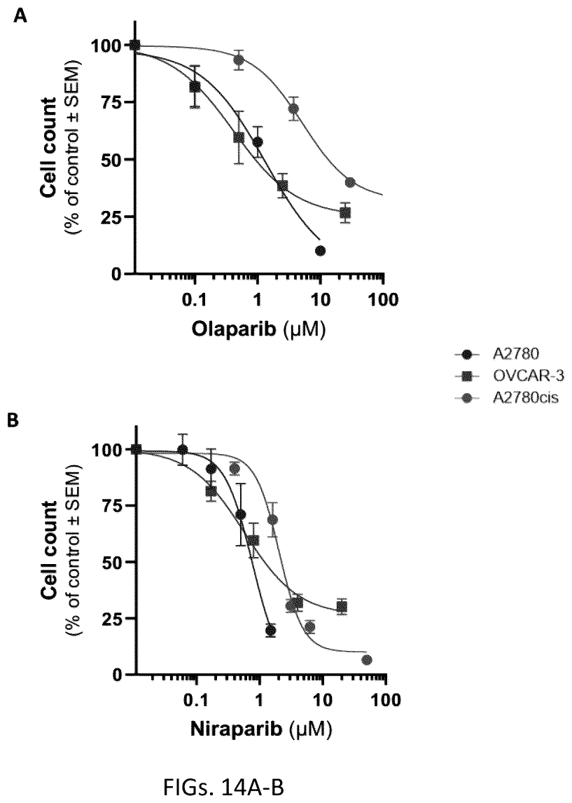

Next, cell count dose response curves of olaparib and niraparib in the three different human ovarian cell lines were measured. As per the case with carboplatin, OVCAR-3 demonstrated highest sensitivity to the PARPi while A2780cis demonstrated highest resistance (FIG. 14). This trait of A2780cis cells suggests that their acquired platinum resistance was also conferring some resistance to PARPi.

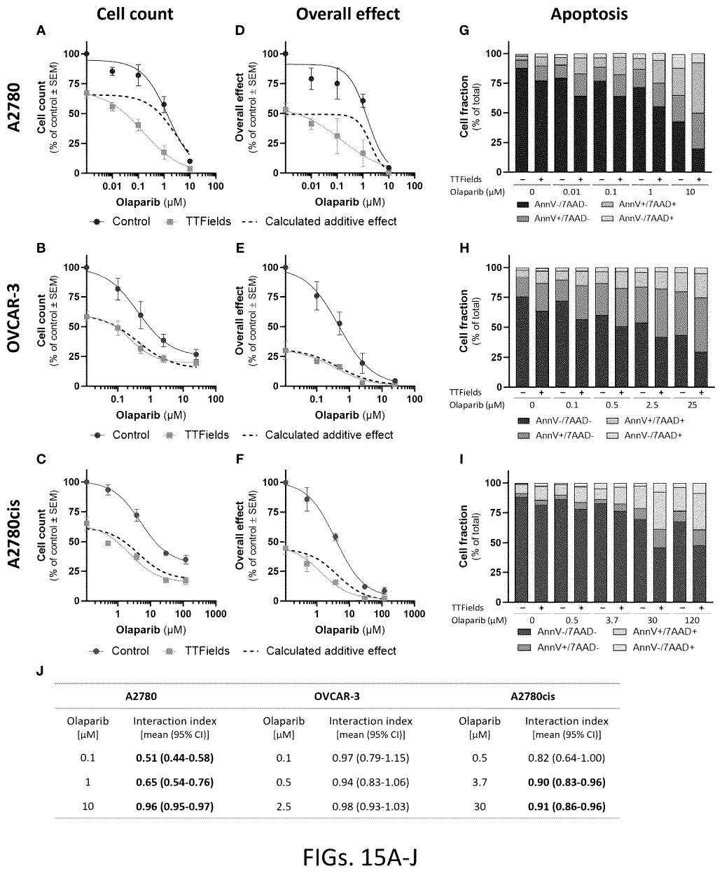

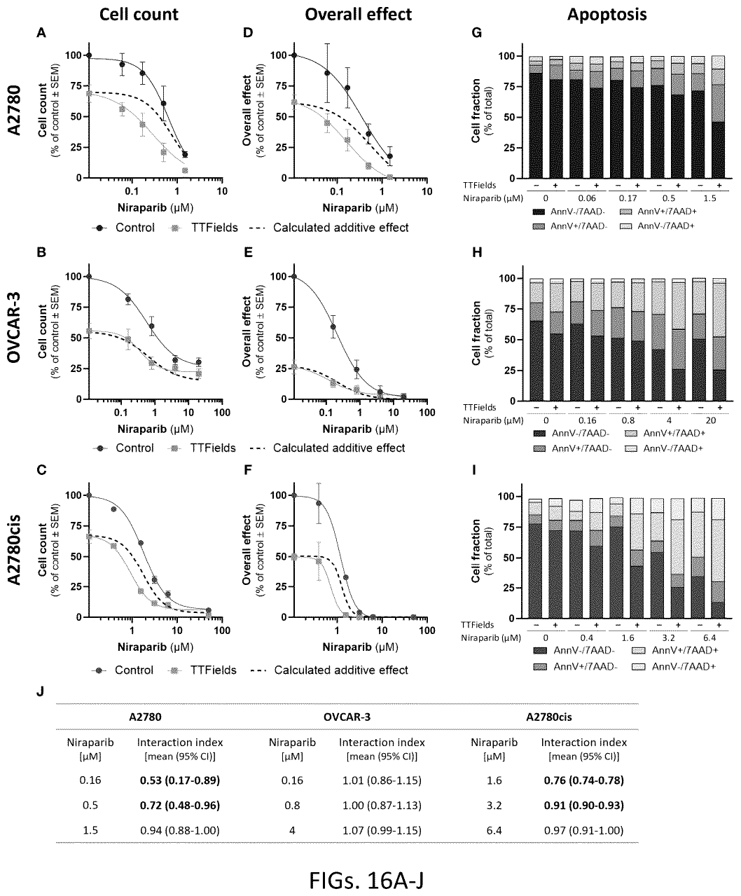

TTFields significantly augmented the effect induced by olaparib and niraparib alone in all examined cell lines, as seen based on cell count (FIGS. 15A-C and FIGS. 16A-C, respectively; p<0.0001 for all cell lines with both drugs) and based on the overall effect (FIGS. 15D-F and FIGS. 16D-F, respectively). Apoptosis analysis demonstrated elevation in the apoptotic cell fraction when TTFields were co-applied with olaparib or niraparib, suggesting a cytotoxic effect (FIGS. 15G-I and FIGS. 16G-I, respectively p<0.0001 for all cell lines with both drugs).

Additivity was determined for TTFields with PARPi in OVCAR-3 cells, as the actual measured curves overlapped the calculated additive curves for the co-treatment (and 95% CI for Iᵢ spanning 1) (FIGS. 15A-F and FIGS. 16A-F, dashed lines; FIG. 15J and FIG. 16J). Different levels of synergy were determined in A2780 and A2780cis cells, with the lower Iᵢ determined for former indicating higher levels of TTFields plus PARPi synergy in A2780 cells. Interestingly, the levels of synergy for TTFields plus PARPi in the A2780 cells was higher than that demonstrated with carboplatin in either A2780 or A2780cis cells.

### TTFields Increase DNA Damage Induced by Carboplatin and PARPi, Downregulate the FA-BRCA Pathway, and Support Drug-Facilitated G2/M Cell Cycle Arrest in Response to the Induced DNA Damage

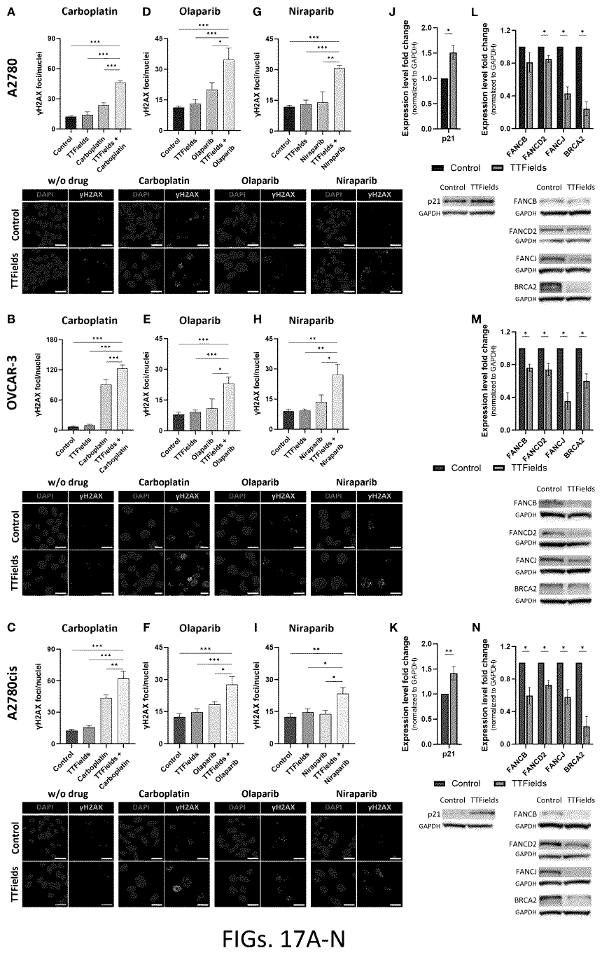

The accumulation of DNA damage in treated cells was examined by fluorescence microscopy detection of γH2AX foci in cell nuclei (FIGS. 17A-I). For these experiments, per each cell line, drugs were used at concentrations that induce 70 to 80 percent reduction in cell count when co-applied with TTFields. Under the selected conditions olaparib and niraparib alone, induced only a mild elevation in the levels of γH2AX in all cell lines. Carboplatin facilitated a more pronounced effect that was especially dramatic in the OVCAR-3 cells. Application of TTFields alone to the cells induced low or no effect on the level of γH2AX foci formation relative to control. However, co-application of TTFields together with either of the three drugs elevated the foci levels significantly relative to control and to TTFields or drug monotherapy.

To further understand how TTFields were involved in elevating DNA damage, possible changes in expression levels of the cyclin-dependent kinase inhibitor p21 (FIGS. 17J-K), a key mediator of DNA damage-induced cell cycle arrest, and of proteins from the FA-BRCA pathway were examined (FIGS. 17L-N). TTFields application to the various cell lines elevated expression levels of p21 in A2780 and A2780cis cells (p21 was not detected in the OVCAR-3 cells, and decreased the expression of FANCB, FANCD2, FANCJ and BRCA2 relative to control cells in all three cell lines.

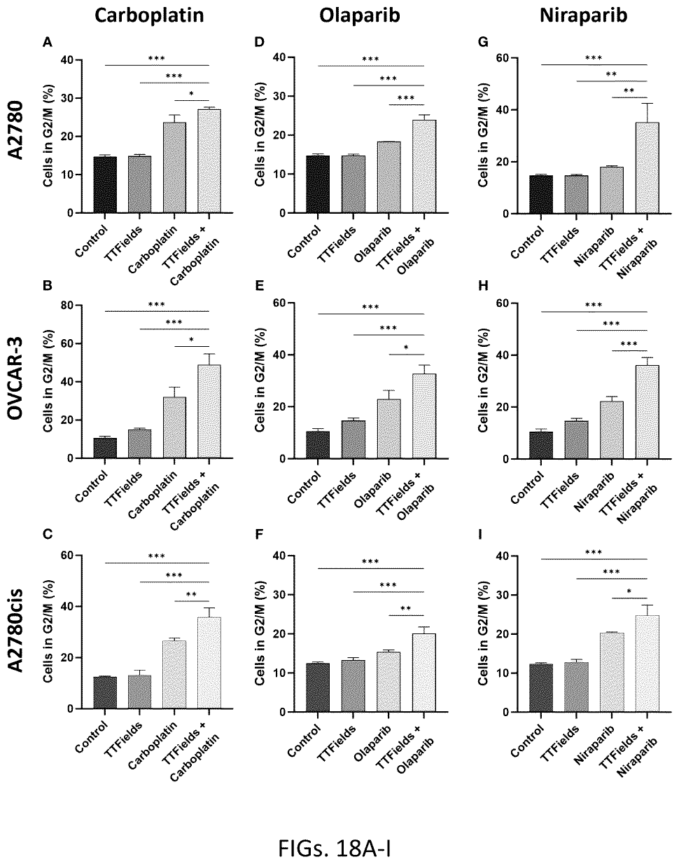

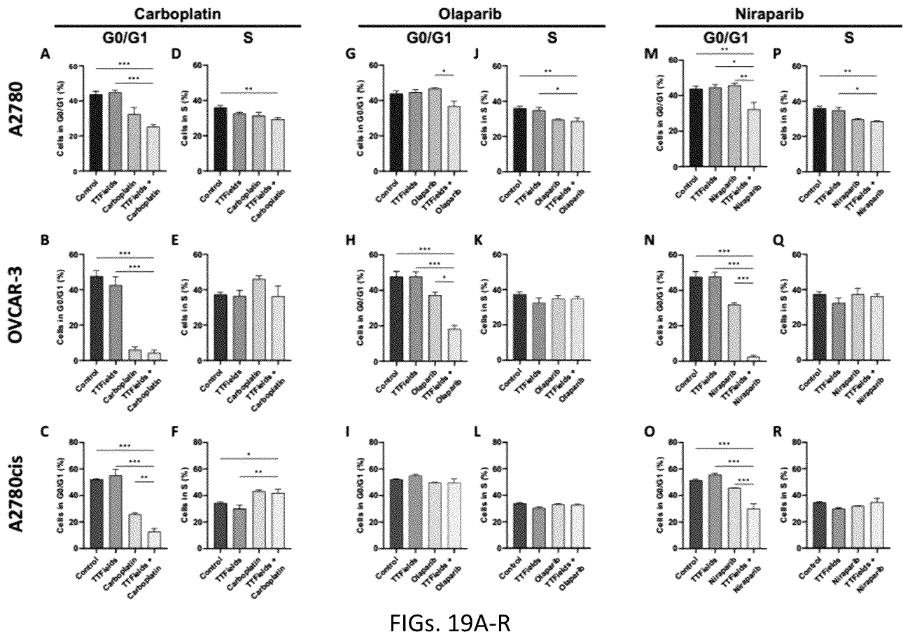

Next, the various treatments were evaluated for their consequent DNA damage formation and for p21 mediated induction of cell cycle arrest (FIGS. 18A-I and FIGS. 19A-R). Carboplatin, olaparib, and niraparib alone all induced G2/M arrest, while co-application of TTFields with each of the drugs significantly elevated the fraction of cells in G2/M.

TTFields Co-Treatment with Olaparib Inhibits Tumor Growth and Prolongs Survival in Ovarian Cancer Bearing Mice

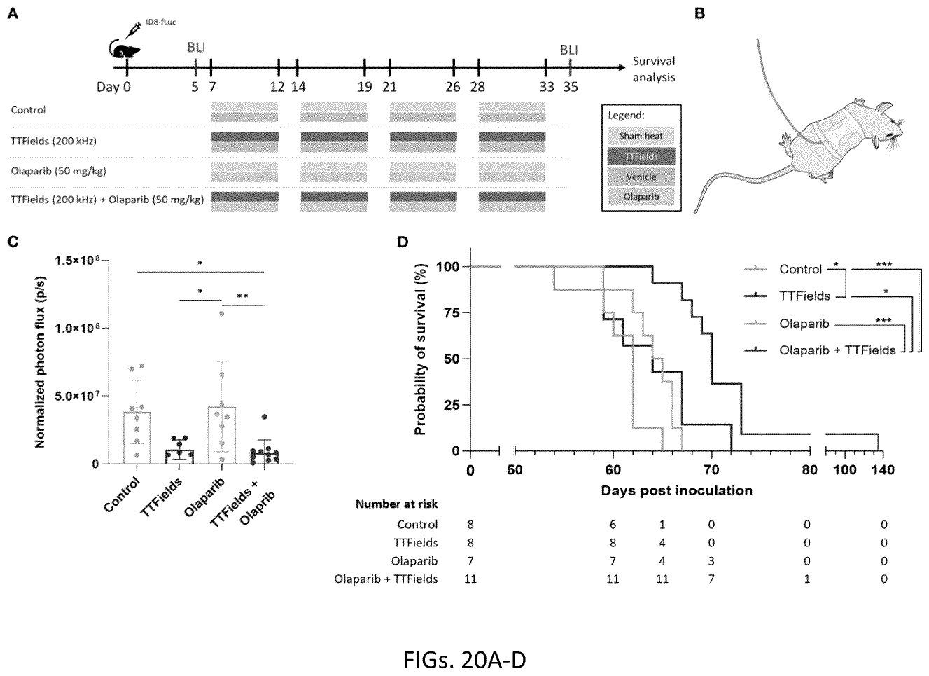

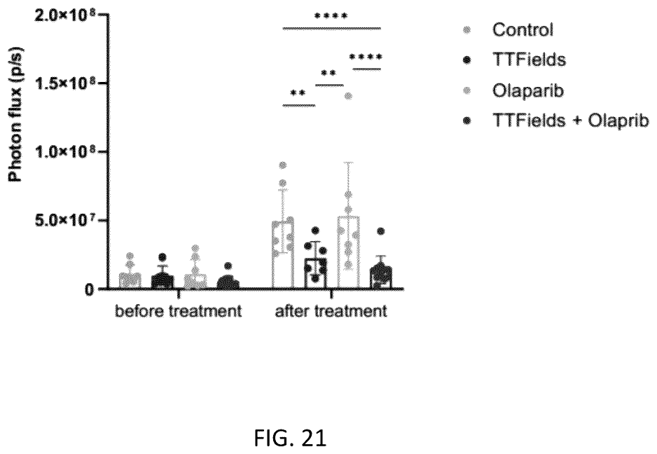

The effect of TTFields application together with olaparib was measured in mice bearing orthotopic ID8-fLuc (HRP) ovarian cancer tumors. Experimental timeline and a schematic illustration of the TTFields/sham arrays attached to the mouse torso are depicted in FIGS. 20A and 20B, respectively. The photon flux measured before treatment showed 100% tumor engraftment, and no significant difference in tumor volume between treatment groups at the start timepoint (FIG. 21). After the 4 weeks treatment period, significantly smaller tumor volumes were seen for mice treated with TTFields plus olaparib, which were lower by about 80% compared to controls (p=0.0183) and to olaparib only treated mice (p=0.0066) (FIG. 20C). Additionally, about 70% reduction in tumor growth was observed in mice treated with TTFields alone compared to olaparib monotherapy (p=0.0288) and to control (p=0.0658).

The Kaplan-Meier curve showed median overall survival of 62 days for control, 64 days for TTFields alone, 64.5 days for olaparib alone, and 70 days for TTFields plus olaparib (FIG. 20D). Survival was significantly prolonged in the mice co-treated with TTFields plus olaparib compared to control mice (p=0.0003), olaparib monotherapy (p=0.0003), and TTFields monotherapy (p=0.0130). Notably, survival benefit was also observed in olaparib only treated mice compared to control mice (p=0.0401).

### 4. Discussion

The efficacy of TTFields for treatment of ovarian cancer has previously been demonstrated in preclinical models and in the INNOVATE study. In those studies, co-application of TTFields with paclitaxel was tested, as to take advantage of the antimitotic effects manifested by both treatment modalities. The effect of TTFields on DNA damage and repair led to examining the effect of adding TTFields to current treatments used for ovarian cancer which target such pathways. Specifically, the effects of TTFields concomitant with carboplatin and PARPi were investigated in HRP, HRD, and platinum-resistant cells.

While TTFields enhanced the efficacy of all the drugs tested in this study in all the examined cell lines, the nature of the interaction was found to be dependent on the drug's underlying mechanism of action, as well as on the genetic background of the cells. In HRD cells (OVCAR-3), carboplatin interacted with TTFields antagonistically, while PARPi interacted with TTFields additively. In HRP cells (A2780) and in platinum-resistant cells (A2780cis), however, all drugs interacted with TTFields synergistically. TTFields-carboplatin synergy was higher in A2780cis relative to A2780 cells, whereas synergy for TTFields with either of the two PARPi (olaparib and niraparib) was higher in A2780 relative to A2780cis cells. Additionally, TTFields with PARPi reached higher levels of synergy relative to TTFields with carboplatin. Overall, these results suggest a potential benefit for concurrent application of TTFields with carboplatin or PARPi in ovarian cancer.

Treatment of the cells with TTFields plus carboplatin or PARPi demonstrated elevated levels of DNA damage, increased expression of p21, a CDK inhibitor involved in induction of cell cycle arrest in response to DNA damage, and elevated G2/M cell cycle arrest. To shed light on this outcome, the effects of TTFields on the expression of proteins from the FA-BRCA pathway were tested. Indeed, TTFields application resulted in decreased expression of FANCB, a protein involved in the FA core complex, FANCD2 from the FANCI-FANCD2 (ID) complex, and of the two pivotal downstream proteins FANCJ and BRCA2, suggesting that the FA-BRCA pathway was severely impaired. While this effect was seen in all the examined cell lines, the manifestation of the effect was dependent on the co-applied drug and the genetic background of the cells.

The differences in the interactions between TTFields and the drugs seen for the different cell lines could be explained based on the HRP/HRD status of the cells. In the HRP cells (A2780), applying TTFields induced a state of BRCAness, hence creating synthetic lethality with PARPi, resulting in a highly synergistic effect. However, in the HRD cells (OVCAR-3), synthetic lethality with PARPi stemmed from the genetic background of the cells, and so the added effect of TTFields on FA-BRCA protein downregulation was transparent. Still, an additive effect for TTFields with PARPi was demonstrated in the HRD cells (OVCAR-3), which may relate to effects of TTFields on cancer cells unrelated to DNA damage repair mechanisms, such as the antimitotic effect. Additionally, TTFields have been shown in GBM cells to increase cell membrane permeability, an effect that was suggested to increase cellular drug uptake, and warrant further examination.

The resolution of DNA damage induced by carboplatin involves multiple factors from different repair pathways, mainly the FA-BRCA pathway and nucleotide excision repair (NER). Synergy between TTFields and carboplatin in the HRP cells (A2780) may therefore be related to this treatment regimen inducing damage while simultaneously blocking one of the pathways needed for its repair. Such cellular conditional vulnerability, with synergy between TTFields and cisplatin, has previously been demonstrated in pleural mesothelioma. The conditional vulnerability instated by the TTFields-induced BRCAness state is however not limited to platinum-based agents, and can also be exploited for concomitant use with other cancer treatment modalities that induce DNA damage. The benefit of applying TTFields with radiation was already demonstrated preclinically, and the potential of this treatment option in patients with newly-diagnosed GBM is currently under clinical investigation.

Platinum resistance is a strong predictive marker for PARPi resistance, indicating inter-related mechanisms. Indeed, the platinum-resistant cell line used in this study (A2780cis) also demonstrated PARPi resistance relative to its parental cell line. Drug resistance mechanisms are complex, encompassing changes in cellular availability of the drugs and alternation in DNA damage response. Changes in the capacity to repair DNA damage were previously demonstrated for A2780 cells resistant to cisplatin compared to their parental cells. Therefore, modulation of DNA damage repair can potentially account for the differences observed in the interaction of TTFields and the drugs between the resistant and the parental A2780 cell lines used in this study. The observation that TTFields application to resistant cells could sensitize them to platinum-based chemotherapy and PARPi is shown herein

The in vivo experiments described herein show the application of TTFields plus olaparib to ovarian cancer HRP cells, the case which showed highest benefit in the in vitro setting. Results showed that TTFields were effective in reducing ID8 tumor growth relative to control, while olaparib was not. When TTFields and olaparib were applied together, significant reduction in tumor growth was observed relative to treatment with olaparib alone and to control. Surprisingly, while TTFields plus olaparib showed similar reduction in tumor growth as TTFields alone, a significant improvement relative to the monotherapies was observed when TTFields were applied together with olaparib in regard to overall survival, confirming the beneficial effect of this treatment regimen.

PARPi have been shown to have immunostimulating mechanisms, including activation of the cGAS/STING pathway in cancer cells. TTFields were also shown to induce immunogenic cell death and cGAS/STING activation in preclinical models and in the clinic.

In conclusion, platinum-based chemotherapy and PARP inhibition are effective mainly in patients with HRD tumors, while patients with HRP tumors show treatment resistance. TTFields induce an HRD-like phenotype, manifesting synergy with the aforementioned drugs, showing potential for ovarian cancer treatment throughout the adjuvant and maintenance stages, in both HRP and HRD ovarian cancer cells, as well as in cells with treatment resistance. As a-priori and de novo drug resistance are a major limitation in ovarian cancer treatment, TTFields-induced sensitization of HRP cells and cells with acquired drug resistance can thus potentially help mitigate the problem.

### Illustrative Embodiments

Embodiment 1 describes a method of enhancing the effect of a platinum-based systemic therapy in a subject having cancer comprising applying an alternating electric field to a target site of the subject for a period of time, the alternating electric field having a frequency and field strength, wherein the target site comprises one or more cancer cells, and administering a therapeutically effective amount of a platinum-based systemic therapy to the subject, wherein the cancer is resistant to platinum-based systemic therapy.

Embodiment 2 describes embodiment 1, wherein the cancer has progressed on or after treatment with a platinum-based systemic therapy

Embodiment 3 describes any one of embodiments 1-2, wherein the subject has been diagnosed with radiological progression while on or after platinum-based systemic therapy.

Embodiment 4 describes any one of embodiments 1-3, wherein the platinum-based systemic therapy that the cancer is resistant to is edaplatin, satraplatin, cisplatin, carboplatin, oxaliplatin, phenanthriplatin, picoplatin, or triplatin tetranitrate.

Embodiment 5 describes any one of embodiments 1-4, wherein the therapeutically effective amount of a platinum-based systemic therapy is a therapeutically effective amount of Nedaplatin, Satraplatin, cisplatin, carboplatin, oxaliplatin, phenanthriplatin, picoplatin, or triplatin tetranitrate.

Embodiment 6 describes embodiment 5, wherein the therapeutically effective amount of a platinum-based systemic therapy is carboplatin.

Embodiment 7 describes any one of embodiments 1-6, wherein the cancer is also resistant to PARP inhibitor treatment prior to applying an alternating electric field and administering a therapeutically effective amount of a platinum-based systemic therapy.

Embodiment 8 describes embodiment 7, wherein the cancer is resistant to carboplatin and a PARP inhibitor.

Embodiment 9 describes any one of embodiments 1-8, further comprising administering a therapeutically effective amount of a PARP inhibitor to the subject.

Embodiment 10 describes embodiment 9, wherein the PARP inhibitor is olaparib, niraparib, talazoparib, or rucaparib.

Embodiment 11 describes a method of enhancing the effect of a PARP inhibitor treatment in a subject having cancer comprising applying an alternating electric field to a target site of the subject for a period of time, the alternating electric field having a frequency and field strength, wherein the target site comprises one or more cancer cells, and administering a therapeutically effective amount of a PARP inhibitor to the subject, wherein the cancer is resistant to platinum-based systemic therapy.

Embodiment 12 describes a method of enhancing the effect of a PARP inhibitor treatment in a subject having cancer comprising applying an alternating electric field to a target site of the subject for a period of time, the alternating electric field having a frequency and field strength, wherein the target site comprises one or more cancer cells, and administering a therapeutically effective amount of a PARP inhibitor to the subject, wherein the cancer is resistant to a PARP inhibitor.

Embodiment 13 describes any one of embodiments 11-12, wherein the cancer has progressed on or after treatment with a platinum-based systemic therapy or PARP inhibitor.

Embodiment 14 describes any one of embodiments 11-13, wherein the subject has been diagnosed with radiological progression while on or after platinum-based systemic therapy or PARP inhibitor.

Embodiment 15 describes any one of embodiments 11-14, wherein the platinum-based systemic therapy that the cancer is resistant to is docetaxel, vinorelbine, nedaplatin, paclitaxel, atraplatin gemcitabine, cisplatin, carboplatin, or oxaliplatin.

Embodiment 16 describes any one of embodiments 11-15, wherein the PARP inhibitor is olaparib, niraparib, talazoparib, or rucaparib.

Embodiment 17 describes any one of embodiments 11-16, wherein the cancer is also resistant to PARP inhibitor treatment prior to applying an alternating electric field and administering a platinum-based systemic therapy or PARP inhibitor.

Embodiment 18 describes embodiment 17, wherein the cancer is resistant to carboplatin and a PARP inhibitor.

Embodiment 19 describes any one of embodiments 11-18, further comprising administering a therapeutically effective amount of a platinum-based systemic therapy to the subject.

Embodiment 20 describes any one of embodiments 1-19, wherein the subject is homologous recombination deficient.

Embodiment 21 describes any one of embodiments 1-20, wherein the cancer has a BRCA mutation.

Embodiment 22 describes any one of embodiments 1-19, wherein the cancer is homologous recombination proficient.

Embodiment 23 describes any one of embodiments 1-19 and 22, wherein the cancer has wild type BRCA.

Embodiment 24 describes any one of embodiments 1-23, wherein the cancer is ovarian cancer, breast cancer, pancreatic cancer, cervical cancer, or brain cancer.

Embodiment 25 describes any one of embodiments 1-24, wherein the target site comprises one or more ovarian cancer cells, breast cancer cells, pancreatic cancer cells, cervical cancer cells, or brain cancer cells.

Embodiment 26 describes any one of embodiments 1-25, wherein the alternating electric field is applied before, after, or simultaneously with administering the platinum-based systemic therapy.

Embodiment 27 describes any one of embodiments 1-26 wherein the alternating electric field is applied before, after, or simultaneously with administering the PARP inhibitor.

Embodiment 28 describes any one of embodiments 1-27, wherein the frequency of the alternating electric field is between 100 kHz and 1 MHz.

Embodiment 29 describes any one of embodiments 1-28 wherein the frequency of the alternating electric field is 200 kHz.

Embodiment 30 describes any one of embodiments 1-29 wherein the alternating electric field has a field strength of between 0.5 and 4 V/cm RMS.

Embodiment 31 describes any one of embodiments 1-30, wherein the alternating electric field has a field strength of 1 V/cm RMS.

Embodiment 32 describes any one of embodiments 1-31, wherein apoptosis of cancer cells in the target site of the subject is increased or wherein cell proliferation of cancer cells in the target site of the subject is decreased.

Embodiment 33 describes methods of treating a subject having cancer comprising applying an alternating electric field to a target site of the subject for a period of time, the alternating electric field having a frequency and field strength, wherein the target site comprises one or more cancer cells, and administering a therapeutically effective amount of a platinum-based systemic therapy to the subject, wherein the cancer is resistant to platinum-based systemic therapy.

Embodiment 34 describes embodiment 33, wherein the cancer is further resistant to PARP inhibitor treatment.

Embodiment 35 describes any one of embodiments 33-34, further comprising administering a PARP inhibitor to the subject.

Embodiment 36 describes methods of treating a subject having cancer comprising applying an alternating electric field to a target site of the subject for a period of time, the alternating electric field having a frequency and field strength, wherein the target site comprises one or more cancer cells, and administering a therapeutically effective amount of a PARP inhibitor to the subject, wherein the cancer is resistant to platinum-based systemic therapy, resistant to a PARP inhibitor, or both.

Embodiment 37 describes embodiment 36, wherein the cancer is further resistant to PARP inhibitor treatment.

Embodiment 38 describes any one of embodiments 36-37, further comprising administering a platinum-based systemic therapy to the subject.

Embodiment 39 describes methods of increasing apoptosis in a cell comprising applying an alternating electric field to a cancer cell for a period of time, the alternating electric field having a frequency and field strength, and administering a therapeutically effective amount of a platinum-based systemic therapy to the cell, wherein the cancer cell is resistant to platinum-based systemic therapy.

Embodiment 40 describes embodiment 39, wherein the cancer cell is further resistant to PARP inhibitor treatment.

Embodiment 41 describes any one of embodiments 39-40, further comprising administering a PARP inhibitor to the cancer cell.

Embodiment 42 describes methods of increasing apoptosis in a cancer cell comprising applying an alternating electric field to a cancer cell for a period of time, the alternating electric field having a frequency and field strength, and administering a therapeutically effective amount of a PARP inhibitor to the cell, wherein the cancer cell is resistant to platinum-based systemic therapy, resistant to a PARP inhibitor, or both.

Embodiment 43 describes embodiment 42, wherein the cancer cell is further resistant to PARP inhibitor treatment.

Embodiment 44 describes any one of embodiments 42-43, further comprising administering a platinum-based systemic therapy to the cancer cell.

Embodiment 45 describes any one of embodiments 33-44, wherein the alternating electric field is applied before, after, or simultaneously with administering the platinum-based systemic therapy.

Embodiment 46 describes any one of embodiments 33-45 wherein the alternating electric field is applied before, after, or simultaneously with administering the PARP inhibitor.

Embodiment 47 describes any one of embodiments 33-46, wherein the frequency of the alternating electric field is between 100 kHz and 1 MHz.

Embodiment 48 describes any one of embodiments 33-47 wherein the frequency of the alternating electric field is 200 kHz.

Embodiment 49 describes any one of embodiments 33-48 wherein the alternating electric field has a field strength of between 0.5 and 4 V/cm RMS.

Embodiment 50 describes any one of embodiments 33-49, wherein the alternating electric field has a field strength of 1 V/cm RMS.

Embodiment 51 describes a kit comprising one or more of platinum-based systemic therapy and one or more materials for delivering an alternating electric field.

Embodiment 52 describes embodiment 51 further comprising a PARP inhibitor.

Embodiment 53 describes a combination of alternating electric fields and a platinum-based systemic therapy for use in enhancing the effect of a platinum-based systemic therapy in a subject having cancer, wherein the cancer is resistant to platinum-based systemic therapy.

Embodiment 54 describes embodiment 53, wherein the cancer has progressed on or after treatment with a platinum-based systemic therapy

Embodiment 55 describes any one of embodiments 53-54, wherein the subject has been diagnosed with radiological progression while on or after platinum-based systemic therapy.

Embodiment 56 describes any one of embodiments 53-55, wherein the platinum-based systemic therapy that the cancer is resistant to is nedaplatin, satraplatin, cisplatin, carboplatin, oxaliplatin, phenanthriplatin, picoplatin, or triplatin tetranitrate.

Embodiment 57 describes any one of embodiments 53-56, wherein the therapeutically effective amount of a platinum-based systemic therapy is a therapeutically effective amount of nedaplatin, satraplatin, cisplatin, carboplatin, oxaliplatin, phenanthriplatin, picoplatin, or triplatin tetranitrate.

Embodiment 58 describes embodiment 57, wherein the therapeutically effective amount of a platinum-based systemic therapy is carboplatin.

Embodiment 59 describes any one of embodiments 53-58, wherein the cancer is also resistant to PARP inhibitor treatment prior to applying an alternating electric field and administering a therapeutically effective amount of a platinum-based systemic therapy.

Embodiment 60 describes embodiment 59, wherein the cancer is resistant to carboplatin and a PARP inhibitor.

Embodiment 61 describes any one of embodiments 53-60, further comprising administering a therapeutically effective amount of a PARP inhibitor to the subject.

Embodiment 62 describes embodiment 61, wherein the PARP inhibitor is olaparib, niraparib, talazoparib, or rucaparib.

Embodiment 63 describes a combination of alternating electric fields and a PARP inhibitor for use in enhancing the effect of a PARP inhibitor in a subject having cancer, wherein the cancer is resistant to platinum-based systemic therapy.

Embodiment 64 describes a combination of alternating electric fields and a PARP inhibitor for use in enhancing the effect of a PARP inhibitor in a subject having cancer, wherein the cancer is resistant to a PARP inhibitor.

Embodiment 65 describes any one of embodiments 63-64, wherein the cancer has progressed on or after treatment with a platinum-based systemic therapy or PARP inhibitor.

Embodiment 66 describes any one of embodiments 63-65, wherein the subject has been diagnosed with radiological progression while on or after platinum-based systemic therapy or PARP inhibitor.

Embodiment 67 describes any one of embodiments 63-66, wherein the platinum-based systemic therapy that the cancer is resistant to is docetaxel, vinorelbine, nedaplatin, paclitaxel, satraplatin gemcitabine, cisplatin, carboplatin, or oxaliplatin.

Embodiment 68 describes any one of embodiments 63-67, wherein the PARP inhibitor is olaparib, niraparib, talazoparib, or rucaparib.

Embodiment 69 describes any one of embodiments 63-68, wherein the cancer is also resistant to PARP inhibitor treatment prior to applying an alternating electric field and administering a platinum-based systemic therapy or PARP inhibitor.

Embodiment 70 describes embodiment 69, wherein the cancer is resistant to carboplatin and a PARP inhibitor.

Embodiment 71 describes any one of embodiments 63-70, further comprising administering a therapeutically effective amount of a platinum-based systemic therapy to the subject.

Embodiment 72 describes any one of embodiments 63-71, wherein the cancer is homologous recombination deficient.

Embodiment 73 describes any one of embodiments 63-72, wherein the cancer has a BRCA mutation.

Embodiment 74 describes any one of embodiments 63-73, wherein the cancer is homologous recombination proficient.

Embodiment 75 describes any one of embodiments 63-74, wherein the cancer has wild type BRCA.

Embodiment 76 describes any one of embodiments 63-75, wherein the cancer is ovarian cancer, breast cancer, pancreatic cancer, cervical cancer, or brain cancer.

Embodiment 77 describes any one of embodiments 63-76, wherein the target site comprises one or more ovarian cancer cells, breast cancer cells, pancreatic cancer cells, cervical cancer cells, or brain cancer cells.

Embodiment 78 describes any one of embodiments 63-77, wherein the alternating electric field is applied before, after, or simultaneously with administering the platinum-based systemic therapy.

Embodiment 79 describes any one of embodiments 63-78 wherein the alternating electric field is applied before, after, or simultaneously with administering the PARP inhibitor.

Embodiment 80 describes any one of embodiments 63-79, wherein the frequency of the alternating electric field is between 100 kHz and 1 MHz.

Embodiment 81 describes any one of embodiments 63-80 wherein the frequency of the alternating electric field is 200 kHz.

Embodiment 82 describes any one of embodiments 63-81 wherein the alternating electric field has a field strength of between 0.5 and 4 V/cm RMS.

Embodiment 83 describes any one of embodiments 63-82, wherein the alternating electric field has a field strength of 1 V/cm RMS.

Embodiment 84 describes any one of embodiments 63-83, wherein apoptosis of cancer cells in the target site of the subject is increased or wherein cell proliferation of cancer cells in the target site of the subject is decreased.

Embodiment 85 describes a combination of alternating electric fields and a platinum-based systemic therapy for use in the treatment of a subject, wherein the cancer is resistant to platinum-based systemic therapy.

Embodiment 86 describes embodiment 85, wherein the cancer is further resistant to PARP inhibitor treatment.

Embodiment 87 describes any one of embodiments 85-86, further comprising administering a PARP inhibitor to the subject.

Embodiment 88 describes a combination of alternating electric fields and a PARP inhibitor for use in the treatment of a subject, wherein the subject is resistant to platinum-based systemic therapy, resistant to a PARP inhibitor or both.

Embodiment 89 describes embodiment 88, wherein the cancer is further resistant to PARP inhibitor treatment.

Embodiment 90 describes any one of embodiments 88-89, further comprising administering a platinum-based systemic therapy to the subject.

Embodiment 91 describes a combination of alternating electric fields and a platinum-based systemic therapy for use in increasing apoptosis in a cancer cell or a subject having cancer, wherein the cell or cancer is resistant to platinum-based systemic therapy.

Embodiment 92 describes embodiment 91, wherein the cell or cancer is further resistant to PARP inhibitor treatment.

Embodiment 93 describes any one of embodiments 91-92, further comprising administering a PARP inhibitor to the subject.

Embodiment 94 describes a combination of alternating electric fields and a PARP inhibitor for use in increasing apoptosis in a cancer cell or a subject having cancer, wherein the cell or cancer is resistant to platinum-based systemic therapy, resistant to a PARP inhibitor, or both.

Embodiment 95 describes embodiment 94, wherein the cancer is further resistant to PARP inhibitor treatment.

Embodiment 96 describes any one of embodiments 94-95, further comprising administering a platinum-based systemic therapy to the subject

Embodiment 97 describes any one of embodiments 85-96, wherein the alternating electric field is applied before, after, or simultaneously with administering the platinum-based systemic therapy.

Embodiment 98 describes any one of embodiments 85-97 wherein the alternating electric field is applied before, after, or simultaneously with administering the PARP inhibitor.

Embodiment 99 describes any one of embodiments 85-98, wherein the frequency of the alternating electric field is between 100 kHz and 1 MHz.

Embodiment 100 describes any one of embodiments 85-99 wherein the frequency of the alternating electric field is 200 kHz.

Embodiment 101 describes any one of embodiments 85-100 wherein the alternating electric field has a field strength of between 0.5 and 4 V/cm RMS.

Embodiment 102 describes any one of embodiments 85-101, wherein the alternating electric field has a field strength of 1 V/cm RMS.

Those skilled in the art will recognize, or be able to ascertain using no more than routine experimentation, many equivalents to the specific embodiments of the method and compositions described herein. Such equivalents are intended to be encompassed by the following claims.

We claim:

1. A method of enhancing the effect of a platinum-based systemic therapy in a subject having cancer comprising:
a. applying an alternating electric field to a target site of the subject for a period of time, the alternating electric field having a frequency and field strength, wherein the target site comprises one or more cancer cells, and
b. administering a therapeutically effective amount of a platinum-based systemic therapy to the subject.
wherein the cancer is resistant to platinum-based systemic therapy.

2. The method of claim 1, wherein the cancer has progressed on or after treatment with a platinum-based systemic therapy.

3. The method of claim 1, wherein the subject has been diagnosed with radiological progression while on or after platinum-based systemic therapy.

4. The method of claim 1, wherein the platinum-based systemic therapy that the cancer is resistant to is nedaplatin, satraplatin, cisplatin, carboplatin, or oxaliplatin.

5. The method of claim 1, wherein the therapeutically effective amount of a platinum-based systemic therapy is a therapeutically effective amount of nedaplatin, satraplatin, cisplatin, carboplatin, or oxaliplatin.

6. The method of claim 1, wherein the cancer is also resistant to PARP inhibitor treatment prior to steps a) and b).

7. The method of claim 1, further comprising c) administering a therapeutically effective amount of a PARP inhibitor to the subject.

8. The method of claim 7, wherein the PARP inhibitor is olaparib, niraparib, talazoparib, or rucaparib.

9. A method of enhancing the effect of a PARP inhibitor treatment in a subject having cancer comprising:
a. applying an alternating electric field to a target site of the subject for a period of time, the alternating electric field having a frequency and field strength, wherein the target site comprises one or more cancer cells, and
b. administering a therapeutically effective amount of a PARP inhibitor to the subject.
wherein the cancer is resistant to platinum-based systemic therapy.

10. The method of claim 9, wherein the cancer has progressed on or after treatment with a platinum-based systemic therapy.

11. The method of claim 9, wherein the subject has been diagnosed with radiological progression while on or after platinum-based systemic therapy.

12. The method of claim 9, wherein the platinum-based systemic therapy that the cancer is resistant to is nedaplatin, satraplatin cisplatin, carboplatin, or oxaliplatin.

13. The method of claim 9, wherein the PARP inhibitor is olaparib, niraparib, talazoparib, or rucaparib.

14. The method of claim 9, wherein the cancer is also resistant to PARP inhibitor treatment prior to steps a) and b).

15. The method of claim 14, wherein the cancer is resistant to carboplatin and a PARP inhibitor.

16. The method of claim 1, wherein the cancer is ovarian cancer, breast cancer, pancreatic cancer, cervical cancer, or brain cancer.

17. The method of claim 1, wherein the target site comprises one or more ovarian cancer cells, breast cancer cells, pancreatic cancer cells, cervical cancer cells, or brain cancer cells.

18. The method of claim 1, wherein the alternating electric field has a frequency of between 100 kHz and 1 MHz, and/or wherein the alternating electric field has a field strength of between 0.5 and 4 V/cm RMS.

19. The method of claim 1, wherein apoptosis of cancer cells in the target site of the subject is increased or wherein cell proliferation of cancer cells in the target site of the subject is decreased.

20. A method of treating a subject having cancer comprising:
applying an alternating electric field to a target site of the subject for a period of time, wherein the target site comprises one or more cancer cells, and
administering a therapeutically effective amount of a platinum-based systemic therapy and/or a PARP inhibitor to the subject.
wherein the cancer is resistant to platinum-based systemic therapy.
